(19) United States
(12) Patent Application Publication
(10) Pub. No.: US 2013/0160169 A1
(43) Pub. Date: Jun. 20, 2013

(54) FUNGAL DESATURASE AND ELONGASE GENES

(71) Applicant: Xiao QIU, Saskatoon (CA)

(72) Inventor: Xiao QIU, Saskatoon (CA)

(73) Assignees: THE UNIVERSITY OF SASKATCHEWAN, Saskatoon (CA); THE GOVERNORS OF THE UNIVERSITY OF ALBERTA, Edmonton (CA)

(21) Appl. No.: 13/330,158

(22) Filed: Dec. 19, 2011

(51) Int. Cl. C12P 7/64; C12N 15/52; C12N 15/63; C12N 15/81; C12N 15/80; C12N 5/10; C12N 15/85; C12N 1/21; C12N 1/19; C12N 1/15; A01H 5/00; A01H 5/10; C12N 15/53; C12N 15/82

(52) U.S. Cl. 800/320.1; 536/23.2; 435/320.1; 435/252.33; 435/254.21; 435/254.3; 435/254.23; 435/252.31; 800/298; 435/419; 800/320; 800/320.3; 435/412; 435/134

(57) ABSTRACT

The invention is directed to isolated polynucleotide and polypeptides of the CoD5, CoD6 and CoE6 genes from Conidiobolus obscurus, the CtE6 gene from Conidiobolus thromboids, and the PgDesX gene from Puccinia graminis; nucleic acid constructs, vectors and host cells incorporating the polynucleotide sequences; and methods of producing and using same.

### FIELD OF THE INVENTION

The present invention is directed to isolated fungal desaturase and elongase genes and gene products; nucleic acid constructs, vectors and host cells incorporating the nucleic acid constructs; and methods of producing and using same.

### BACKGROUND OF THE INVENTION

Conidiobolus fungi are mainly found to inhabit soil or decaying plant materials in tropical areas, particularly in areas near the equator such as Africa, India and Central America. There are over twenty-one known species within the genus, some of which have been found to be causative agents in human infections, but the fungal species Conidiobolus obscurus is known to strictly infect insects (Scorsetti et al., 2007). As C. obscurus is particularly fond of the aphid host, it has been used as bio-pesticides in controlling the aphid population in various crops such as potato, small grain and cotton (Feng et al., 1990; Milner and Soper, 1981; Steinkraus and Tugwell, 1997). This fungus is also able to produce substantial amounts of very long chain polyunsaturated fatty acids (VLCPUFAs) (Tyrrell, 1967).

Polyunsaturated fatty acids have been shown to play an important role in sexual development and spore germination of several filamentous fungi. In Neurospora sp., α-linolenic acid (ALA) stimulates formation of fruiting bodies (Nukina et al., 1981). In Mucor sp., γ-linolenic acid (GLA) is steadily increased during the germination process of spores (Laoteng et al., 2000) where the Δ6 desaturase gene responsible for the biosynthesis of the fatty acid is highly expressed (Khunyoshyeng et al., 2002). When the fungus infects the host, it produces yeast-like hyphal bodies and wall-less protoplasts. The protoplasts, unlike hyphal bodies, are not recognized by the immune system of insects because of the lack of beta-1,3 glucan in the cell walls (Tanada and Kaye, 2003). It appears that VLCPUFAs can inhibit the synthesis of beta-1,3 glucan in the protoplasts, allowing the fungus to evade the host immune system and to eventually kill its host (Mackichan et al., 1995).

VLCPUFAs such as arachidonic acid (ARA, 20:4n-6), eicosapentaenoic acid (EPA, 20:5n-3) and docosahexaenoic acid (DHA, 22:6n-3) are essential fatty acids for human health. Dietary supplementations of these VLCPUFAs may provide protection against many chronic diseases and enhance eye and brain function (Napier, 2006; Ratledge and Wynn, 2002; Venegas-Caleron et al., 2010).

Fatty acid desaturases catalyze dehydrogenation reactions resulting in the introduction of double bonds into fatty acid chains (Sperling et al., 2003). Desaturases can be classified into two main groups according to the enzyme solubility. Soluble desaturases such as acyl-ACP (acyl carrier protein) desaturases are found in the plant chloroplast stroma, which convert saturated fatty acyl-ACPs to their monounsaturated counterparts, whereas membrane-bound desaturases are more widely spread in nature, and use acyl-lipid or acyl-CoA as substrates (Shanklin and Cahoon, 1998). The ω-3 desaturases are membrane-bound enzymes involved in the biosynthesis of ω-3 PUFAs from ω-6 fatty acids. Most known ω-3 desaturases desaturate linoleic acid (LA) to ALA, but cannot desaturate 20-carbon PUFAs (Tocher et al., 1998). Caenorhabditis elegans w-3 desaturase expressed in yeast acts on fatty acid chain lengths from 18 to 20. However the activity towards the 20-carbon PUFAs was quite low (Meesapyodsuk et al., 2000). The fungus pathogen Claviceps purpurea ω-3 desaturase can desaturate fatty acids from carbon 18 to 20, predominantly desaturated 18-carbon fatty acids such as LA and GLA (Meesapyodsuk et at, 2007).

The biosynthesis of VLCPUFAs mostly proceeds with the Δ6 desaturation pathway in eukaryotes. For instance, ω-3 VLCPUFAs are synthesized in most fungi first through sequential Δ12 and ω-3 desaturations of oleic acid (18:1-9) resulting in ALA (18:3n-3), which is followed by Δ6 desaturation and Δ6 elongation giving rise to eicosatetraenoic acid (ETA, 20:4n-3). ETA is desaturated by a Δ5 desaturase producing EPA. The biosynthesis of ω6 VLCPUFAs occurs in a similar process. The Δ6 desaturation of linoleic acid (LA, 18:2n-6) results in GLA (18:3n-6), which is followed by Δ6 elongation and Δ5 desaturation producing arachidonic acid (ARA, 20:4-5,8,11,14).

ETA (20:4-8,11,14,17) is a ω-3 VCLPUFA that has recently attracted scientific attention for its unique chemical properties and biological activities, and being the precursor for the biosynthesis of downstream ω-3 VLCPUFAs.

Currently, the main source of the ω-3 VLCPUFAs for human dietary supplements is marine fish. However, with the steady declining fish population in oceans, there is a need to find alternative sources for these fatty acids to meet the growing demand. Accordingly, there is a need in the art for renewable, cost-effective sources of VCLPUFAs, which may be used as human dietary supplements or animal feeds.

### SUMMARY OF THE INVENTION

In one aspect, the present invention relates to CoD5, CoD6 and CoE6 genes from Conidiobolus obscurus, the CtE6 gene from Conidiobolus thromboids, and the PgDesX gene from Puccinia graminis.

In one aspect, the invention comprises an isolated polynucleotide encoding a polypeptide having Δ5, M, or ω-3 desaturase activity or Δ6 elongase activity and comprising:
1. an amino acid sequence selected from SEQ ID NO: 2, 4, 6, 8 or 10; or
2. an amino acid sequence having at least 85% sequence identity with one of SEQ ID NO: 2, 4, 6, 8 or 10.

In one embodiment, the polynucleotide encodes a polypeptide having Δ5 desaturase activity and comprising the amino acid sequence of SEQ ID NO: 2 or an amino acid sequence having at least 85% sequence identity therewith.

In one embodiment, the polynucleotide encodes a polypeptide having Δ6 desaturase activity and comprising the amino acid sequence of SEQ ID NO: 4 or an amino acid sequence having at least 85% sequence identity therewith.

In one embodiment, the polynucleotide encodes a polypeptide having ω-3 desaturase activity and comprising the amino acid sequence of SEQ ID NO: 10 or an amino acid sequence having at least 85% sequence identity therewith.

In one embodiment, the polynucleotide encodes a polypeptide having Δ6 elongase activity and comprising the amino acid sequence of SEQ ID NO: 6 or 8, or an amino acid sequence having at least 85% sequence identity with one of SEQ ID NO: 6 or 8.

In one embodiment, the polynucleotide comprises a nucleotide sequence of SEQ ID NO: 1, 3, 5, 7 or 9, or a nucleotide sequence having at least 85%, at least 90%, at least 95%, at least 96%, at least 97%, at least 98%, or at least 99% sequence identity to one of SEQ ID NOS: 1, 3, 5, 7 and 9, and encoding an enzyme having Δ5, Δ6, or ω-3 desaturase activity or Δ6 elongase activity, and.

In one embodiment, the encoded polypeptide has Δ5, Δ6, or ω-3 desaturase activity or Δ6 elongase activity, and comprises an amino acid sequence having at least 90%, at least 95%, at least 96%, at least 97%, at least 98%, or at least 99% sequence identity to SEQ ID NO: 2, 4, 6, 8 or 10.

In one embodiment, the polynucleotide is derived from Conidiobolus obscurus, Conidiobolus thromboids, or Puccinia graminis.

In another aspect, the invention comprises a polynucleotide construct or a vector comprising a polynucleotide as described herein, operably linked to a promoter expressible in bacterial, yeast, fungal, mammalian or plant cells.

In another aspect, the invention comprises a microbial cell comprising the above polynucleotide. In one embodiment, the cell is Saccharomyces cerevisiae, Aspergillus, Pichia pastoris, Escherichia coli or Bacillus subtilis.

In another aspect, the invention comprises a transgenic plant, plant cell, plant seed, callus, plant embryo, microspore-derived embryo, or microspore, comprising the above polynucleotide. In one embodiment, the transgenic plant, plant cell, plant seed, callus, plant embryo, microspore-derived embryo, or microspore is selected from a linseed, canola, peanut, safflower, flax, oats, wheat, triticale, barley, corn, and legume plant, plant cell, plant seed, callus, plant embryo, or microspore-derived embryo or microspore.

In another aspect, the invention comprises a method for producing a transgenic plant comprising the steps of introducing into a plant cell or a plant tissue the above polynucleotide to produce a transformed cell or plant tissue; and cultivating the transformed plant cell or transformed plant tissue to produce the transgenic plant. In one embodiment, the plant is selected from a linseed, canola, peanut, safflower, flax, oats, wheat, triticale, barley, corn, and legume plant, plant cell, plant seed, callus, plant embryo, or microspore-derived embryo or microspore.

In yet another aspect, the invention comprises a method for producing a polyunsaturated fatty acid comprising the steps of:
a) constructing one or more vectors comprising one or more of the above polynucleotides;
b) transforming the one or more vectors into a host cell under conditions sufficient for expression of a Δ5, Δ6 or ω-3 desaturase or a Δ6 elongase encoded by the polynucleotides; and
c) exposing the Δ5, Δ6, or ω-3 desaturase or Δ6 elongase to a fatty acid substrate, wherein the substrate is converted by the Δ5, Δ6, or ω-3 desaturase or Δ6 elongase into a desired polyunsaturated fatty acid product.

In one embodiment, the fatty acid substrate comprises one or more of linoleic acid, γ-linolenic acid, α-linolenic acid, stearidonic acid, dihomo-γ-linolenic acid, arachidonic acid, eicosatetraenoic acid, and eicosapentaenoic acid. In one embodiment, the polypeptide comprises a Δ6 desaturase, and the fatty acid substrate comprises linoleic acid or α-linolenic acid. In one embodiment, the polypeptide comprises a Δ6 elongase, and the fatty acid substrate comprises γ-linolenic acid, stearidonic acid, arachidonic acid or eicosapentaenoic acid. In one embodiment, the polypeptide comprises an ω-3 desaturase, and the fatty acid substrate comprises linoleic acid, γ-linoleic acid, dihomo gamma-linoleic acid and arachidonic acid. In one embodiment, the host cell comprises a bacterial, yeast, fungal, mammalian or plant cell.

In another aspect, the invention comprises a microbial cell which comprises a heterologous eicosatetraenoic acid biosynthetic pathway comprising a Δ6 desaturase, a Δ6 elongase, a Δ12 desaturase and an ω3 desaturase. In one embodiment, the microbial cell comprises CoD6 and CoE6 from C. obscurus, CpDes12 and CpDesX from Claviceps purpurea. In one embodiment, the microbial cell comprises a yeast, such as Saccharomyces cerevisiae.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings:

FIG. 1 shows the fatty acid profile of TAGs and total phospholipids in C. obscurus.

FIGS. 2A-B show the functional analysis of CoD6 in yeast with linoleic acid (18:2-9, 12). FIG. 2A is a gas chromatogram of yeast transformants with pYES2.1 (the control) and pYES2.1-CoD6. Fatty acid peaks: (1) 16:0, (2) 16:1-9, (3) 16:2-6, 9, (4) 18:0, (5) 18:1-9, (6) 18:1-11, (7) 18:2-6, 9, (8) 18:2-9, 12 and (9) 18:3-6,9,12. FIG. 2B is a mass spectrum of Peak 9 in A. FID: flame ionization detector, M⁺: molecular ion of positively charged fatty acids.

FIGS. 3A-B show functional analysis of CoE6 in yeast with stearidonic acid (18:4-6,9,12,15). FIG. 3A is a gas chromatogram of yeast transformants with pYES2.1 (the control) and pYES2.1-CoE6. Fatty acid peaks: (1) 16:0, (2) 16:1-9, (3) 18:0, (4) 18:1-9, (5) 18:1-11, (6) 18:4-6,9,12,15, (7) 20:4-8,11,14,17. FIG. 3B is a mass spectrum of Peak 7 in A.

FIG. 4 shows the transcript levels of CoD6 and CoE6 grown under different temperatures.

FIG. 5 shows reconstitution of the entire ETA pathway in yeast by co-expressing CoD6, CoE6, CpDes12 and CpDesX pESC-HIS/pESC-URA: the control. pESC-HIS-CoD6-CoE6/pESC-URA-CpDes12-CpDesX: the four gene transformant. Fatty acid peaks: (1) 16:0, (2) 16:1-9, (3) 16:2-9, 12, (4) 16:3-9,12,15, (5) 18:0, (6) 18:1-9, (7) 18:1-11, (8) 18:2-9, 12, (9) 18:2-11,14, (10) 18:3-6,9,12, (11) 18:3-9,12,15, (12) 18:4-6,9,12,15, (13) 20:3-8,11,14, (14) 20:3-11,14,17 and (15) 20:4-8,11,14,17.

FIG. 6A shows the CoD5 nucleotide sequence, while FIG. 6B shows the CoD5 amino acid sequence.

FIG. 7A shows the CoD6 nucleotide sequence, while FIG. 7B shows the CoD6 amino acid sequence.

FIG. 8A shows the CoE6 nucleotide sequence, while FIG. 8B shows the CoE6 amino acid sequence.

FIG. 9A shows the CtE6 nucleotide sequence, while FIG. 9B shows the CtE6 amino acid sequence.

FIG. 10A shows the PgDesX nucleotide sequence, while FIG. 10B shows the PgDesX amino acid sequence.

### DETAILED DESCRIPTION OF PREFERRED EMBODIMENTS

The present invention relates to isolated polynucleotides and polypeptides of the CoD5, CoD6 and CoE6 genes from Conidiobolus obscurus, the CtE6 gene from Conidiobolus thromboids, and the PgDesX gene from Puccinia graminis; nucleic acid constructs, vectors and host cells incorporating the polynucleotide sequences; and methods of producing and using same. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims. To facilitate understanding of the invention, the following definitions are provided:

A “cDNA” is a polynucleotide which is complementary to a molecule of mRNA. The “cDNA” is formed of a coding sequence flanked by 5′ and 3′ untranslated sequences.

A “coding sequence” or “coding region” or “open reading frame (ORF)” is part of a gene that codes for an amino acid sequence of a polypeptide.

A “complementary sequence” is a sequence of nucleotides which forms a duplex with another sequence of nucleotides according to Watson-Crick base pairing rules where “A” pairs with “T” and “C” pairs with “G.”

A “construct” is a polynucleotide which is formed by polynucleotide segments isolated from a naturally occurring gene or which is chemically synthesized. The “construct” is combined in a manner that otherwise would not exist in nature, and is usually made to achieve certain purposes. For instance, the coding region from “gene A” can be combined with an inducible promoter from “gene B” so the expression of the recombinant construct can be induced.

“Downstream” means on the 3′ side of a polynucleotide while “upstream” means on the 5′ side of a polynucleotide.

“Expression” refers to the transcription of a gene into RNA (rRNA, tRNA) or messenger RNA (mRNA) with subsequent translation into a protein.

“Gene” means a DNA segment which contributes to phenotype or function, and which may be characterized by sequence, transcription or homology.

“Isolated” means that a substance or a group of substances is removed from the coexisting materials of its natural state.

“Nucleic acid” means polynucleotides such as deoxyribonucleic acid (DNA), and, where appropriate, ribonucleic acid (RNA). The term should also be understood to include, as equivalents, analogs of either RNA or DNA.

As used herein, the term “plasmid” means a DNA molecule which is separate from, and can replicate independently of, the chromosomal DNA. They are double stranded and, in many cases, circular. Plasmids used in genetic engineering are known as vectors and are used to multiply or express particular genes. Any plasmid may be used for the present invention provided that the plasmid contains a gene which encodes a CoD5, CoD6, CoE6, CtE6 and PgDesX, or a variant thereof in an expressible manner. In one embodiment, the plasmid comprises a yeast expression vector. Those skilled in art will recognize that any plasmid in the art may be modified for use in the compositions and methods of the present invention. As used herein, the term “regulatory element” includes, but is not limited to, a promoter, enhancer, terminator, and the like which are required for the expression of the encoded CoD5, CoD6, CoE6, CtE6 and PgDesX, or variant thereof.

A “polynucleotide” is a linear sequence of ribonucleotides (RNA) or deoxyribonucleotides (DNA) in which the 3′ carbon of the pentose sugar of one nucleotide is linked to the 5′ carbon of the pentose sugar of another nucleotide. The deoxyribonucleotide bases are abbreviated as “A” deoxyadenine; “C” deoxycytidine; “G” deoxyguanine; “T” deoxythymidine; “I” deoxyinosine. Some oligonucleotides described herein are produced synthetically and contain different deoxyribonucleotides occupying the same position in the sequence. The blends of deoxyribonucleotides are abbreviated as “W” A or T; “Y” C or T; “H” A, C or T; “K” G or T; “D” A, G or T; “B” C, or T; “N” A, C, G or T.

A “polypeptide” is a sequence of amino acids linked by peptide bonds. Common amino acids referred to herein are abbreviated as “A” alanine; “R” arginine; “N” asparagine; “D” aspartic acid; “C” cysteine; “Q” glutamine; “E” glutamic acid; “G” glycine; “H” histidine; “I” isoleucine; “L” leucine; “K” lysine; “M” methionine; “F” phenylalanine; “P” proline; “S” serine; “T” threonine; “W” tryptophan; “Y” tyrosine and “V” valine.

Two polynucleotides or polypeptides are “identical” if the sequence of nucleotides or amino acids, respectively, in the two sequences is the same when aligned for maximum correspondence as described here. Sequence comparisons between two or more polynucleotides or polypeptides can be generally performed by comparing portions of the two sequences over a comparison window which can be from about 20 to about 200 nucleotides or amino acids, or more. The “percentage of sequence identity” may be determined by comparing two optimally aligned sequences over a comparison window, wherein the portion of a polynucleotide or a polypeptide sequence may include additions (i.e., insertions) or deletions (i.e., gaps) as compared to the reference sequence. The percentage is calculated by determining the positions at which identical nucleotides or identical amino acids are present, dividing by the number of positions in the window and multiplying the result by 100 to yield the percentage of sequence identity. Polynucleotide and polypeptide sequence alignment may be performed by implementing specialized algorithms or by inspection. Examples of sequence comparison and multiple sequence alignment algorithms are: BLAST and ClustalW software. Identity between nucleotide sequences can also be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. Hybridization methods are described in Ausubel et al. (1995).

A “desaturase” is an enzyme that removes two hydrogen atoms from a fatty acid, creating a carbon/carbon double bond. Desaturases are classified as “delta-n” indicating that the double bond is created at a fixed position from the carboxyl group of a fatty acid (for example, a delta-5 desaturase creates a double bond at the fifth position from the carboxyl end, while a delta-6 desaturase creates a double bond at the sixth position from the carboxyl end); or omega indicating the double bond is created between the third and fourth carbon from the methyl end of the fatty acid (for example, an omega-3 desaturase).

An “elongase” is an enzyme that is involved in the elongation of saturated and monounsaturated VLCFAs, or an enzyme which is an elongase of polyunsaturated fatty acids.

A “promoter” is a polynucleotide usually located within 20 to 5000 nucleotides upstream of the initiation of translation site of a gene. The “promoter” determines the first step of expression by providing a binding site to DNA polymerase to initiate the transcription of a gene. The promoter is said to be “inducible” when the initiation of transcription occurs only when a specific agent or chemical substance is presented to the cell. For instance, the GAL “promoter” from yeast is “inducible by galactose,” meaning that this GAL promoter allows initiation of transcription and subsequent expression only when galactose is presented to yeast cells.

A “recombinant” polynucleotide is a novel polynucleotide sequence formed in vitro through the ligation of two DNA molecules.

“Transformation” means the directed modification of the genome of a cell by external application of a polynucleotide, for instance, a construct. The inserted polynucleotide may or may not integrate with the host cell chromosome. For example, in bacteria, the inserted polynucleotide usually does not integrate with the bacterial genome and might replicate autonomously. In plants, the inserted polynucleotide integrates with the plant chromosome and replicates together with the plant chromatin.

A “transgenic” organism is the organism that was transformed with an external polynucleotide. The “transgenic” organism encompasses all descendants, hybrids and crosses thereof, whether reproduced sexually or asexually and which continue to harbor the foreign polynucleotide.

A “vector” is a polynucleotide that is able to replicate autonomously in a host cell and is able to accept other polynucleotides. For autonomous replication, the vector contains an “origin of replication.” The vector usually contains a “selectable marker” that confers the host cell resistance to certain environment and growth conditions. For instance, a vector that is used to transform bacteria usually contains a certain antibiotic “selectable marker” which confers the transformed bacteria resistance to such antibiotic.

The present invention relates to isolated polynucleotides and polypeptides of the CoD5, CoD6 and CoE6 genes from Conidiobolus obscurus, the CtE6 gene from Conidiobolus thromboids, and the PgDesX gene from Puccinia graminis; nucleic acid constructs, vectors and host cells incorporating the polynucleotide sequences; and methods of producing and using same.

In one aspect, the invention provides isolated CoD5, CoD6, CoE6, CtE6 and PgDesX polynucleotides, and polypeptides having Δ5, Δ6, or ω-3 desaturase activity or Δ6 elongase activity. CoD5, CoD6, CoE6, CtE6 and PgDesX polynucleotides include, without limitation (1) single- or double-stranded DNA, such as cDNA or genomic DNA including sense and antisense strands; and (2) RNA, such as mRNA. CoD5, CoD6, CoE6, CtE6 and PgDesX polynucleotides include at least a coding sequence which codes for the amino acid sequence of the specified polypeptide, but may also include 5′ and 3′ untranslated regions and transcriptional regulatory elements such as promoters and enhancers found upstream or downstream from the transcribed region.

In one embodiment, the invention provides a CoD5 polynucleotide which is a cDNA comprising the nucleotide sequence depicted in SEQ ID NO: 1, and which was isolated from Coniodiobolus obscurus. The cDNA comprises a coding region of 1418 base pairs. The delta-5 desaturase encoded by the coding region (designated as CoD5, SEQ ID NO: 2) is a 471 amino acid polypeptide.

In one embodiment, the invention provides a CoD6 polynucleotide which is a cDNA comprising the nucleotide sequence depicted in SEQ ID NO: 3, and which was isolated from Coniodiobolus obscurus. The cDNA comprises a coding region of 1,347 base pairs. The delta-6 desaturase encoded by the coding region (designated as CoD6, SEQ ID NO: 4) is a 449 amino acid polypeptide with a predicted molecular mass of 51.7 kDa. CoD6 introduces a delta-6 double bond into linoleic acid and α-linolenic acid.

In one embodiment, the invention provides a CoE6 polynucleotide which is a cDNA comprising the nucleotide sequence depicted in SEQ ID NO: 5, and which was isolated from Coniodiobolus obscurus. The cDNA comprises a coding region of 984 base pairs. The delta-6 elongase encoded by the coding region (designated as CoE6, SEQ ID NO: 6) is a 328 amino acid polypeptide with a predicted molecular weight of 37.3 kDa. CoE6 elongates 18-carbon delta-6 desaturated fatty acids, such as γ-linolenic acid and stearidonic acid.

In one embodiment, the invention provides a CtE6 polynucleotide which is a cDNA comprising the nucleotide sequence depicted in SEQ ID NO: 7, and which was isolated from Conidiobolus thromboids. The cDNA comprises a coding region of 1254 base pairs. The delta-6 elongase encoded by the coding region (designated as CtE6, SEQ ID NO: 8) is a 329 amino acid polypeptide. CtE6 elongates 18-carbon delta-6 desaturated fatty acids, such as γ-linolenic acid and stearidonic acid, and 20-carbon VLCPUFAs, such as ARA and EPA.

In one embodiment, the invention provides a PgDesX polynucleotide which is a cDNA comprising the nucleotide sequence depicted in SEQ ID NO: 9, and which was isolated from Puccinia graminis. The cDNA comprises a coding region of 1419 base pairs. The omega-3 desaturase encoded by the coding region (designated as PgDesX, SEQ ID NO: 10) is a 472 amino acid polypeptide. PgDesX introduces an omega-3 double bond into linoleic acid (LA, 18:2-9, 12), gamma-linoleic acid (GLA, 18:3,-6,9,12), dihomo gamma-linoleic acid (DGLA, 20:3-8,11,14) and arachidonic acid (AA, 20:4-5,8,11,14), thereby converting these omega-6 polyunsaturated fatty acids into their omega-3 counterparts.

Those skilled in the art will recognize that the degeneracy of the genetic code allows for a plurality of polynucleotides to encode for identical polypeptides. Accordingly, the invention includes polynucleotides of SEQ ID NOS: 1, 3, 5, 7 and 9 and variants of polynucleotides encoding polypeptides of SEQ ID NOS: 2, 4, 6, 8 and 10. In one embodiment, polynucleotides having at least 85%, at least 90%, at least 95%, at least 96%, at least 97%, at least 98%, or at least 99% sequence identity to the nucleotide sequences depicted in SEQ ID NOS: 1, 3, 5, 7 and 9 are included in the invention. Methods for isolation of such polynucleotides are well known in the art (Ausubel et al., 1995).

In one embodiment, the invention provides isolated polynucleotides which encode CoD5, CoD6, CoE6, CtE6 or PgDesX, or polypeptides having amino acid sequences having at least 85%, at least 90%, at least 95%, at least 96%, at least 97%, at least 98%, or at least 99% sequence identity to the amino acid sequences depicted in SEQ ID NOS: 2, 4, 6, 8 and 10.

The above described polynucleotides of the invention may be used to express polypeptides in recombinantly engineered cells including, for example, bacterial, yeast, fungal, mammalian or plant cells. In one embodiment, the invention provides polynucleotide constructs, vectors and cells comprising CoD5, CoD6, CoE6, CtE6 or PgDesX polynucleotides. Those skilled in the art are knowledgeable in the numerous systems available for expression of a polynucleotide. All systems employ a similar approach, whereby an expression construct is assembled to include the coding sequence of interest and control sequences such as promoters, enhancers, and terminators, with signal sequences and selectable markers included if desired. Briefly, the expression of isolated polynucleotides encoding polypeptides is typically achieved by operably linking, for example, the DNA or cDNA to a constitutive or inducible promoter, followed by incorporation into an expression vector. The vectors can be suitable for replication and integration in either prokaryotes or eukaryotes. Typical expression vectors include transcription and translation terminators, initiation sequences, and promoters useful for regulation of the expression of the DNA. High level expression of a cloned gene is obtained by constructing expression vectors which contain a strong promoter to direct transcription, a ribosome binding site for translational initiation, and a transcription/translation terminator. Vectors may further comprise transit and targeting sequences, selectable markers, enhancers or operators. Means for preparing vectors are well known in the art. Typical vectors useful for expression of polynucleotides in plants include for example, vectors derived from the Ti plasmid of Agrobacterium tumefaciens and the pCaM-VCN transfer control vector. Promoters suitable for plant cells include for example, the nopaline synthase, octopine synthase, and mannopine synthase promoters, and the caulimovirus promoters.

Those skilled in the art will appreciate that modifications (i.e., amino acid substitutions, additions, deletions and post-translational modifications) can be made to a polypeptide of the invention without eliminating or substantially diminishing its biological activity. Conservative amino acid substitutions (i.e., substitution of one amino acid for another amino acid of similar size, charge, polarity and conformation) or substitution of one amino acid for another within the same group (i.e., nonpolar group, polar group, positively charged group, negatively charged group) are unlikely to alter protein function adversely. Some modifications may be made to facilitate the cloning, expression or purification. Variant CoD5, CoD6, CoE6, CtE6 and PgDesX polypeptides may be obtained by mutagenesis of the polynucleotides depicted in SEQ ID NOS: 1, 3, 5, 7 and 9 using techniques known in the art including, for example, oligonucleotide-directed mutagenesis, region-specific mutagenesis, linker-scanning mutagenesis, and site-directed mutagenesis by PCR (Ausubel et al., 1995).

Various methods for transformation or transfection of cells are available. For prokaryotes, lower eukaryotes and animal cells, such methods include for example, calcium phosphate precipitation, fusion of the recipient cells with bacterial protoplasts containing the DNA, treatment of the recipient cells with liposomes containing the DNA, DEAE dextran, electroporation, biolistics and microinjection. The transfected cells are cultured, and the produced CoD5, CoD6, CoE6, CtE6 and PgDesX polypeptides may be isolated and purified from the cells using standard techniques known in the art. Various industrial strains of microorganisms including for example, Aspergillus, Pichia pastoris, Saccharomyces cerevisiae, Escherichia coli, Bacillus subtilis may be used to produce CoD5, CoD6, CoE6, CtE6 and PgDesX polypeptides. In one embodiment, the microorganism comprises S. cerevisiae. In one embodiment, the microorganism comprises an oleaginous microorganism. As used herein, an “oleaginous microorganism” accumulates a substantial portion (for example, more than 20%) of its biomass as lipid, usually in the form of triacylglycerols. The oils produced by oleaginous microorganisms are similar to plant oils.

Methods for transformation of plant cells include for example, electroporation, PEG poration, particle bombardment, Agrobacterium tumefaciens- or Agrobacterium rhizogenes-mediated transformation, and microinjection. The transformed plant cells, seeds, callus, embryos, microspore-derived embryos, microspores, organs or explants are cultured or cultivated using standard plant tissue culture techniques and growth media to regenerate a whole transgenic plant which possesses the transformed genotype. Transgenic plants may pass polynucleotides encoding CoD5, CoD6, CoE6, CtE6 and PgDesX polypeptides to their progeny, or can be further crossbred with other species. Accordingly, in one embodiment, the invention provides methods for producing transgenic plants, plant cells, callus, seeds, plant embryos, microspore-derived embryos, and microspores comprising CoD5, CoD6, CoE6, CtE6 and PgDesX polynucleotides.

In one embodiment, the invention provides transgenic plants, plant cells, callus, seeds, plant embryos, microspore-derived embryos, or microspores, comprising CoD5, CoD6, CoE6, CtE6 or PgDesX polynucleotides. Plant species of interest for transformation include, without limitation, oilseeds (for example, the linseed plant, rapeseed or canola, peanut, safflower), flax, oats, wheat, triticale, barley, corn, and legume plants including soybean and pea.

In one embodiment, the invention comprises a method for producing a polyunsaturated fatty acid comprising the steps of
a) constructing one or more vectors comprising one or more of the polynucleotides claimed herein;
b) transforming the one or more vectors into a host cell under conditions sufficient for expression of a Δ5, Δ6 or ω-3 desaturase or a Δ6 elongase encoded by the polynucleotides; and
c) exposing the Δ5, Δ6, or ω-3 desaturase or Δ6 elongase to a fatty acid substrate, wherein the substrate is converted by the Δ5, Δ6, or ω-3 desaturase or Δ6 elongase into a desired polyunsaturated fatty acid product.

The CoD5, CoD6, CoE6, CtE6 and PgDesX isolated polynucleotides and polypeptides of the present invention may be incorporated into human food and animal feed applications to produce health supplements or to improve the nutritional quality of products. For example, a nutraceutical product having a fatty acid profile which reduces the risk of chronic diseases and enhances eye and brain function may be developed for humans.

The following are specific examples of embodiments of the present invention. It will be appreciated by those skilled in the art that the isolated polynucleotide and polypeptides of the CoD5, CoD6 and CoE6 genes from Conidiobolus obscurus, the CtE6 gene from Conidiobolus thromboids, and the PgDesX gene from Puccinia graminis have industrial applications. The biosynthesis of VLCPUFAs involves alternating desaturation and elongation. The CoD5 and CoD6 genes encode delta-5 and delta-6 desaturases, respectively. The CoE6 and CtE6 genes encode delta-6 elongases. The PgDesX gene encodes an omega-3 desaturase. The examples demonstrate how these genes can be used to produce VCLPUFAs for human dietary supplements or animal feeds.

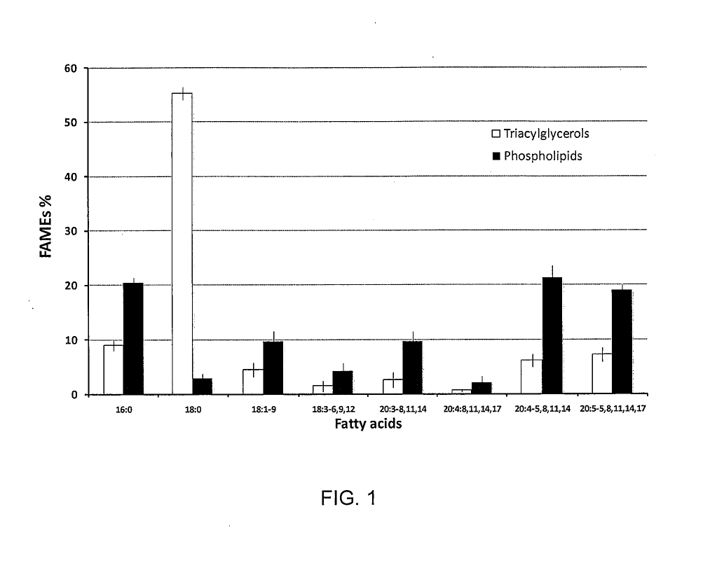

When grown at room temperature, C. obscurus produces substantial amounts of VLCPUFAs in triacylglycerol (TAG) and phospholipid fractions, including both ω-3 and ω6 VLCPUFAs such as EPA (eicosapentaenoic acid, 20:5n-3) and ARA (arachidonic acid, 20:4n-6), and ETA (eicosatetraenoic acid, 20:4n-3) and DGLA (dihomo-γ-linolenic acid, 20:3n-6), respectively. The Δ5 desaturation producing these latter two fatty acids is generally believed to occur in phospholipids (Domergue et al., 2003). Although ARA and EPA are not the predominate fatty acids in the TAG fraction, they were two major fatty acids in the phospholipid fraction, together they made up approximately 40% of the total fatty acids (FIG. 1).

Degenerate RT-PCR and RACE methods were employed to clone genes involved in the biosynthesis of VLCPUFAs from C. obscurus. To clone the gene encoding Δ6 desaturase (CoD6), a pair of degenerate oligonucleotide primers was designed to the well-conserved heme-binding site ((A/E)-(D/K)-H-P-G-G; SEQ ID NO: 11) and the third histidine box (W-F-H-G-G-L-Q; SEQ ID NO: 12) of several Δ6 desaturases previously identified from other eukaryotes. RT-PCR amplification with the degenerate primers using the total RNA as a template generated a cDNA fragment of about ˜1000 bp long which showed high sequence similarity to other Δ6 desaturases. The RACE method was then used to obtain the full-length cDNA (SEQ ID NO: 3). The open reading frame of the full-length cDNA CoD6 was 1,347 nucleotides in length and encoded a 449 amino acid polypeptide with a predicted molecular mass of 51.7 kDa (SEQ ID NO: 4). Comparison of the CoD6 protein with related sequences indicated it had 46%, 42% and 40% of amino acid identity to Δ6 desaturases from M. alpina (Huang et al., 1999), M. circinelloides (Michinaka et al., 2003) and R. stolonifer (Zhang et al., 2004), respectively.

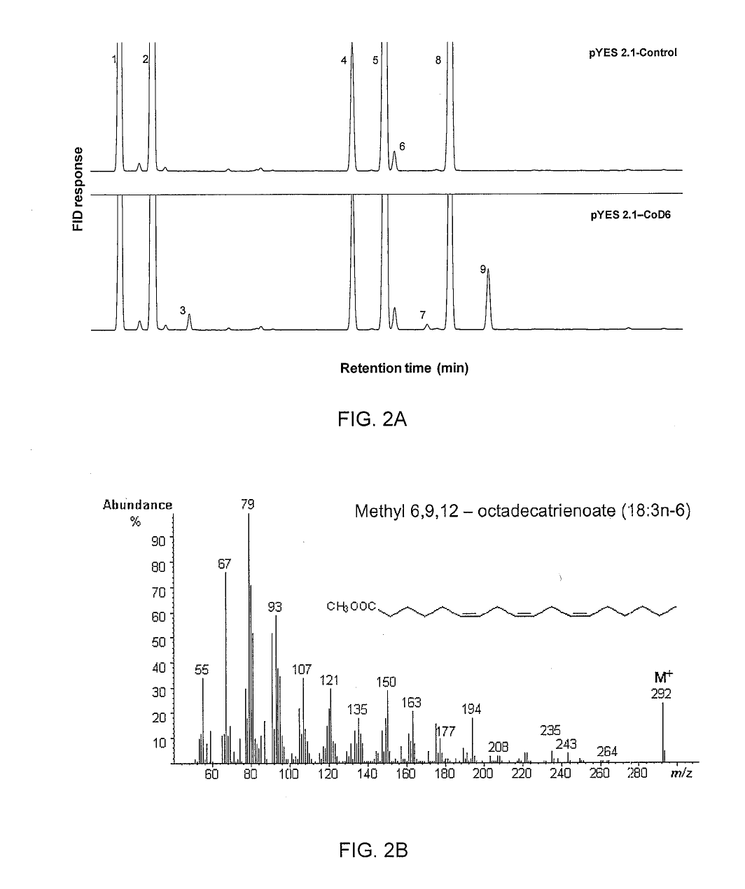

To define the function of CoD6, the ORF was cloned into a yeast expression vector pYES2.1 under the control of GAL1 promoter. The plasmid was transformed into S. cerevisiae INVSc1. Selected transformants were grown in the presence of linoleic acid, a known substrate for Δ6 desaturase. Fatty acid analysis showed that compared with the control, transformants expressing CoD6 in presence of linoleic acid produced a new peak with the retention time and mass spectrum identical to that of γ-linolenic acid (GLA, 18:3-6,9,12) (FIG. 2), indicating CoD6 coded for a functional Δ6 desaturase which could introduce a Δ6 double bond into linoleic acid, giving rise to GLA. The Δ6 desaturase cDNA CoD6, when expressed in yeast, thus introduces a double bond at the sixth position of linoleic acid and α-linolenic acid. However, the desaturase activity appeared to be relatively low in comparison to other previously characterized Δ6 desaturases from fungi.

To determine the substrate specificity of CoD6, transformants were exogenously supplied with several other fatty acids that differed in the number and position of double bonds as well as in chain length. The results from these feeding experiments showed that the highest desaturation activity of CoD6 was detected on LA and ALA and similar desaturation efficiency [(products/(substrates+products)×%] was observed with these two substrates (15% and 16%, respectively). In addition to introducing a Δ6 double bond into LA and ALA, CoD6 could also, to a lesser extent, use 16:1-9 and 18:1-9 as substrates, producing 16:2-6, 9 and 18:2-6, 9, respectively. The Δ6 desaturated products of two preferred substrates, linoleic acid and linolenic acid were in a range of 2 to 3% compared to 10% of M. alpina Δ6 desaturase (Huang et al. 1999), 7.1% of M. rouxii Δ6 desaturase (Laoteng et al. 2000), 6.0% of P. irregulare Δ6 desaturase (Hong et al. 2002) under similar experimental conditions.

The full-length putative Δ5 desaturase cDNA (CoD5) from C. obscures was also cloned. The open reading frames of the cDNAs were inserted into a yeast expression vector (pYES2.1/V5-His-TOPO) and transformed into a yeast strain InvSc1. The activity of these genes was investigated by feeding the transformants with probable substrates and incubating at 20° C. for 3 days. Functional expression of these genes in S. cerevisiae showed one of these genes, CoD5, codes for a functional Δ5 desaturase, which can introduce a Δ5 double bond into DGLA and ETA.

The putative elongase gene from C. obscurus (CoE6) was also cloned. Two conserved regions, the histidine rich motif (F-L-H-V-Y-H-H; SEQ ID NO: 13) and the tyrosine rich motif (M-Y-T-Y-Y-F-L-S; SEQ ID NO: 14) found in several Δ6 elongases previously identified from fungi, algae and animals were used to design two degenerate oligonucleotide primers for RT-PCR. Degenerate RT-PCR produced a ˜150 bp fragment with the total RNA as the template, which showed high sequence similarity to a Δ6 elongase from M. alpina (Parker-Barnes et al., 2000). The RACE method was then used to obtain the full-length cDNA (SEQ ID NO: 5). The open reading frame of the full-length cDNA CoE6 was 984 bp and encoded a polypeptide of 328 amino acids with molecular weight of 37.3 kDa (SEQ ID NO: 6). Comparison of CoE6 with other known Δ6 elongases showed that it had high amino acid identity to elongases from M. alpina (Parker-Barnes et al., 2000) (52%), P. patens (Zank et al., 2000) (41%), O. tauri (Domergue et al., 2005) (40%), Marchantia polymorpha (Kajikawa et al., 2004) (36%) and Thraustochytrium sp. (Wu et al., 2005) (39%).

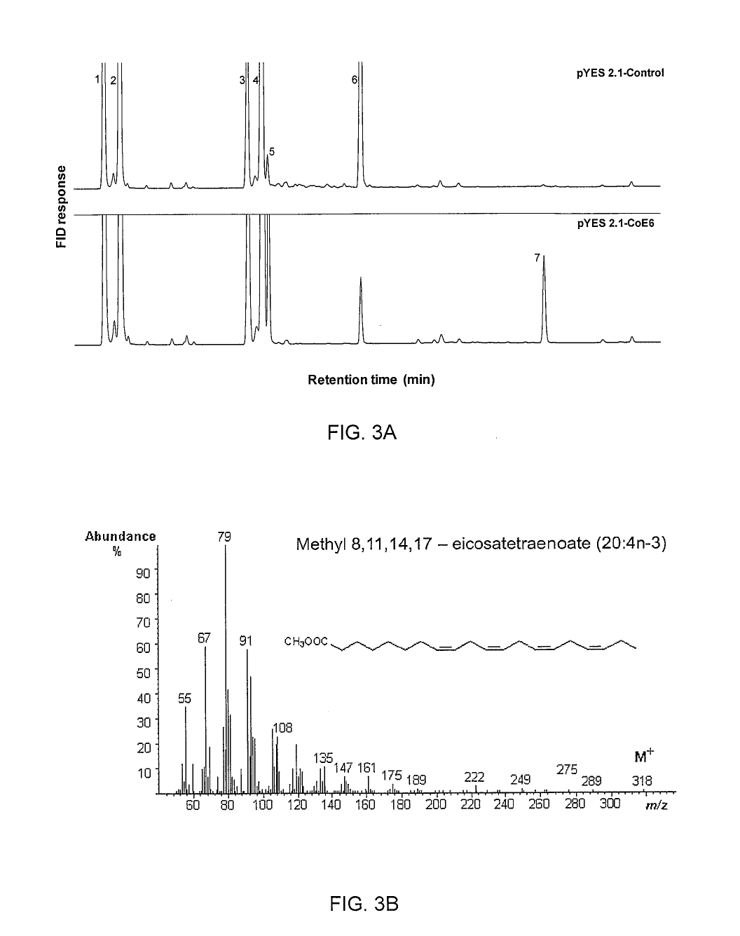

To define the function of CoE6, the ORF was similarly cloned into the yeast expression vector. The selected transformants were grown in the presence of stearidonic acid (SDA, 18:4-6,9,12,15), a known substrate for Δ6 elongase. Fatty acid analysis showed that compared with the control, transformants expressing CoE6 in presence of stearidonic acid produced a new peak with the same retention time as eicosatetraenoic acid (ETA). GC/MS analysis of this peak confirmed it had the same mass spectrum as 20:4-8,11,14,17, indicating that CoE6 encoded a functional elongase that could elongate stearidonic acid to ETA (FIG. 3).

Substrate specificity analysis indicated CoE6 was able to effectively elongate both GLA and SDA with high elongation efficiency (50% and 60%, respectively). SDA was the most preferred substrate producing ETA. Besides, CoE6 could also, at a reduced efficiency, elongate 17:1-10 and 18:3-9,12,15 producing 19:1-12 and 20:3-11,14,17, respectively. However, it could not elongate any very long chain fatty acids (>18 C) such as 20:1-11, 22:1-13, 20:4-5,8,11,14 and 20:5-5,8,11,14,17.

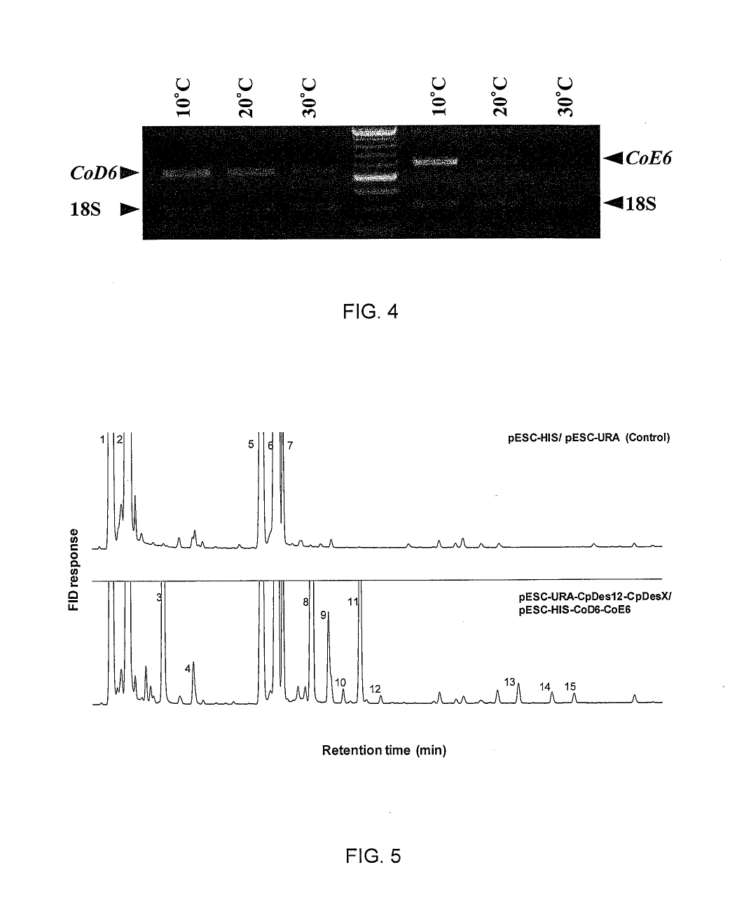

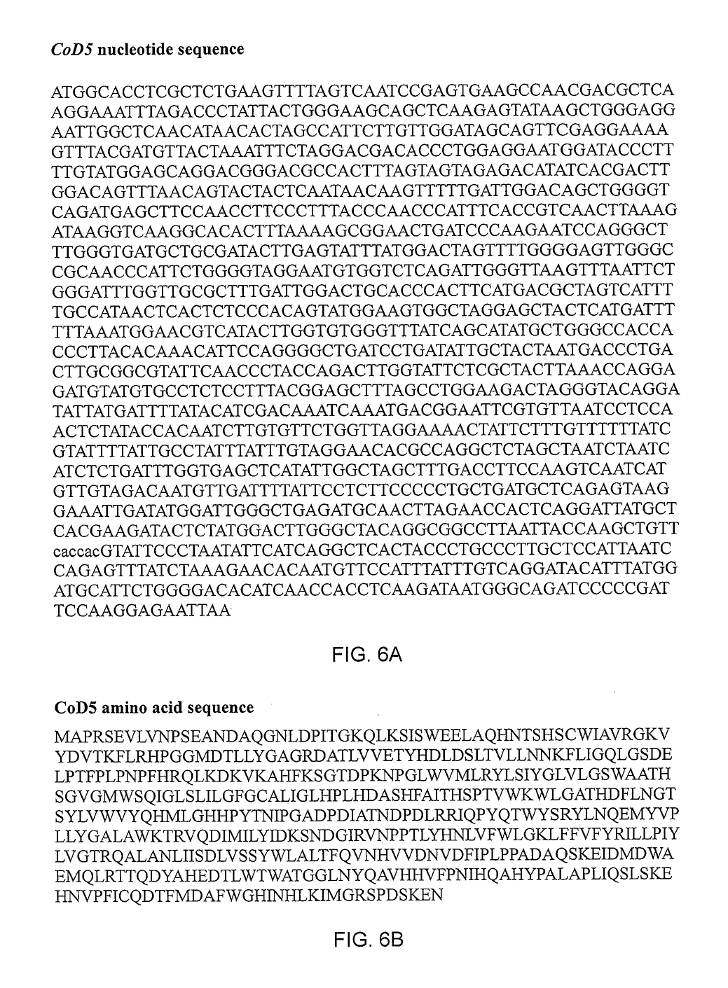

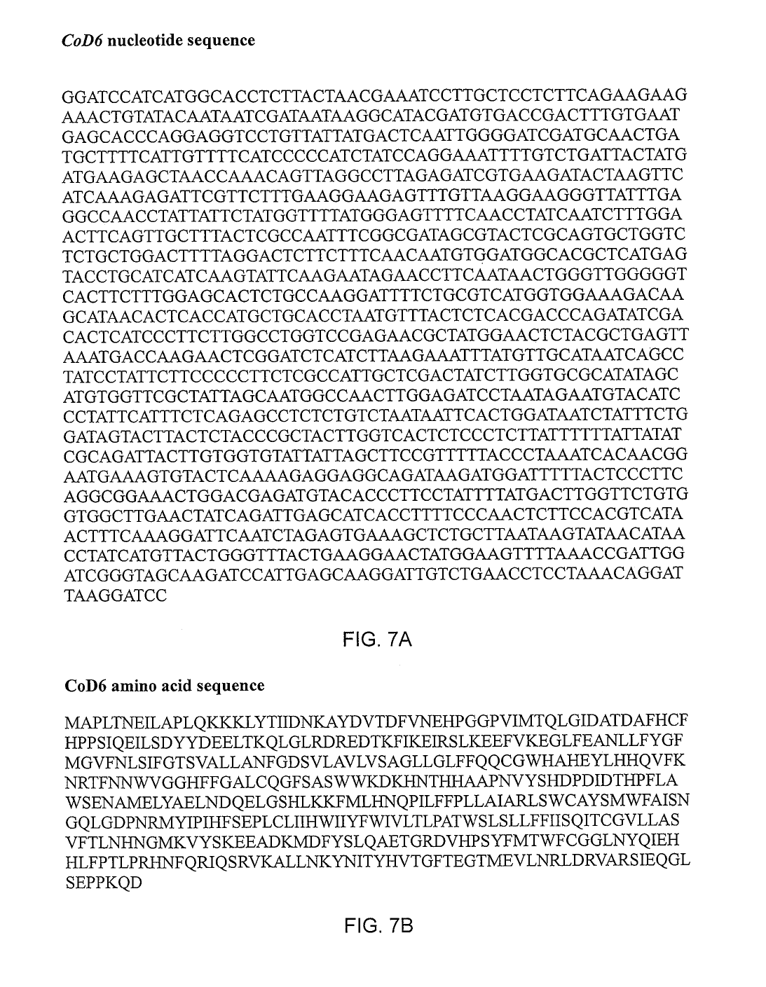

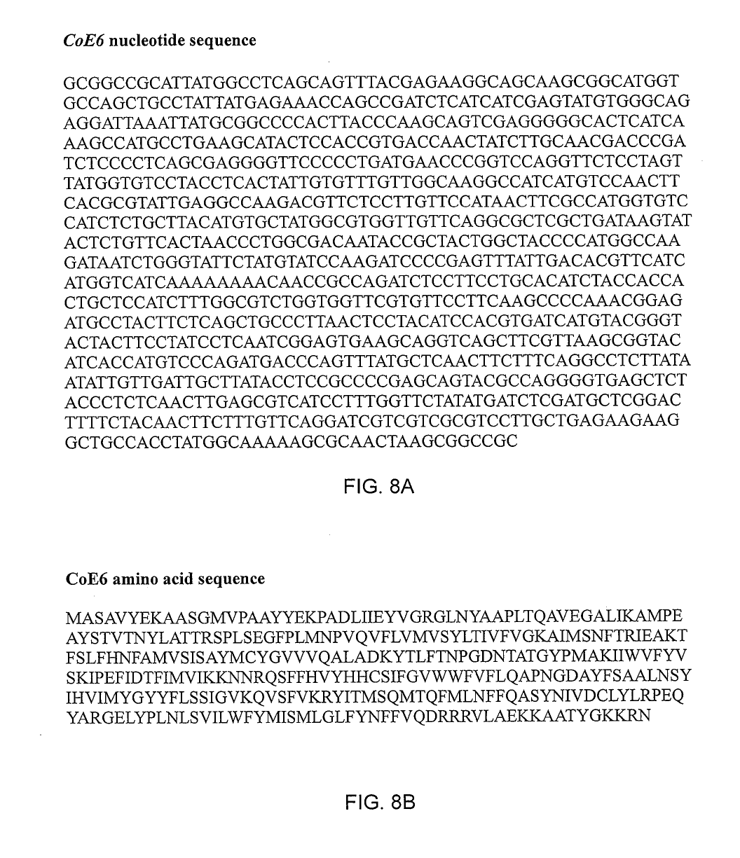

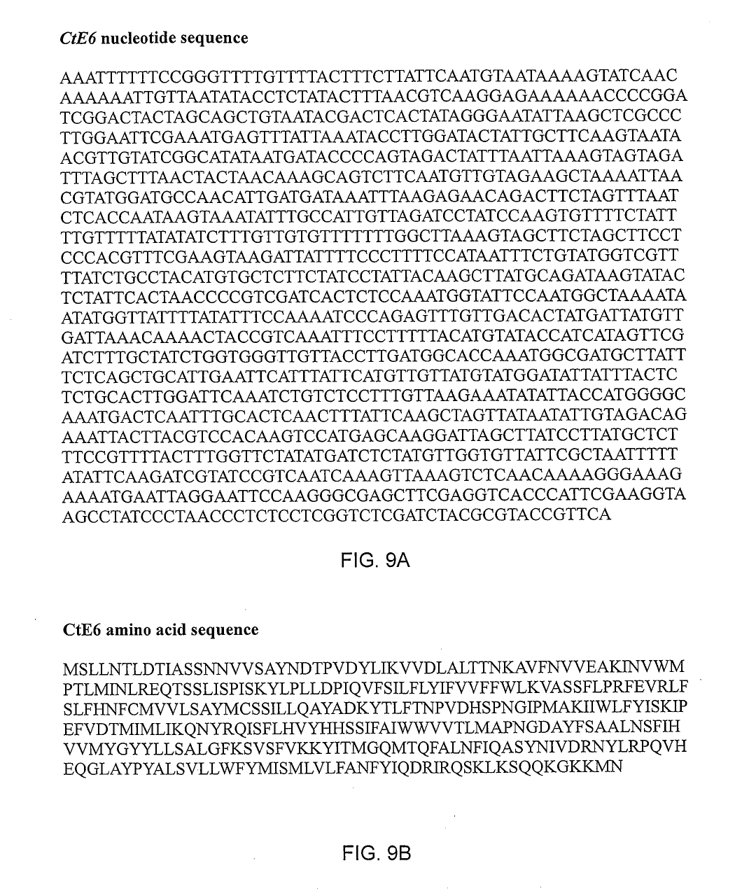

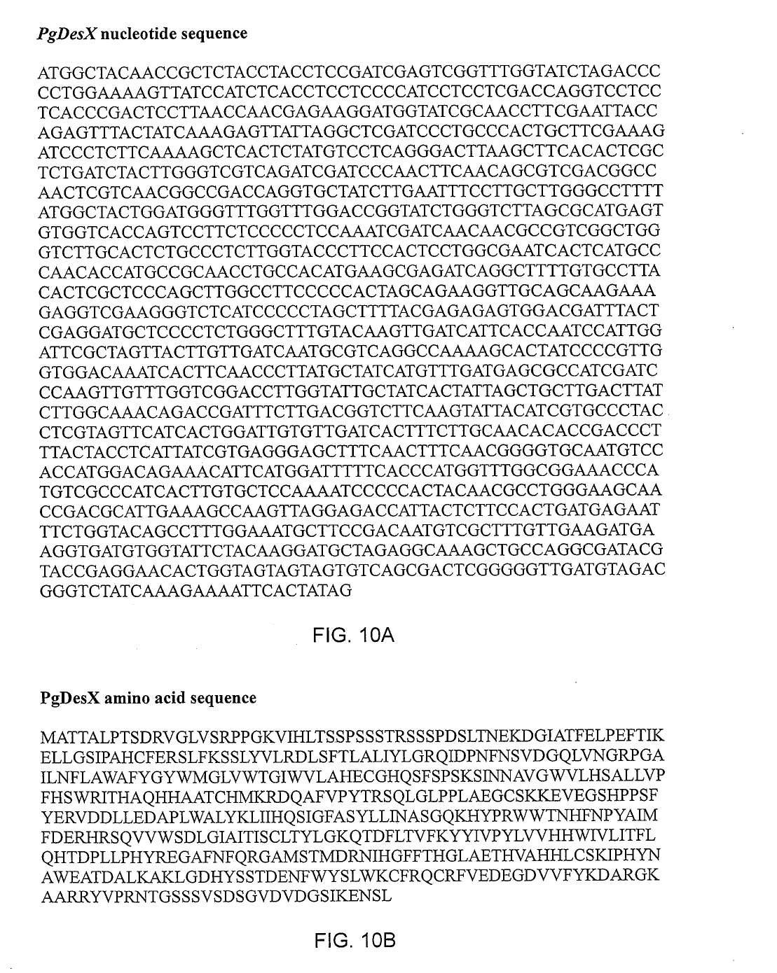

To examine whether the growth temperature has any effect on expression of the two genes, the transcript levels of CoD6 and CoE6 from C. obscurus grown under 10° C., 20° C. and 30° C. were compared through a RT-PCR method based on an internal standard. The result showed that the growth temperature had significant impact on the transcript level of the two genes. When the fungus was grown at 10° C., the transcript levels of both CoD6 and CoE6 were increased, relative to those grown at 20° C. On the other hand, when the fungus was grown at 30° C., the transcript levels of both genes were decreased by dramatically relative to those grown at 20° C. (FIG. 4). It was also noted that the transcript change of the two genes under different temperatures was generally correlated with the alteration of fatty acid composition of the cell total lipids (Table 1).

TABLE 1
The fatty acid composition of the total lipids of C. obscurus grown under different temperatures

| | | | | | | | | | Δ6 | Δ 6 |
|---|---|---|---|---|---|---|---|---|---|---|
| | | | | | | | | | desaturation | elongation |
| Temp | LA | GLA | ALA | SDA | DGLA | ARA | ETA | EPA | efficiency | efficiency |
| 10° C. | 2.84 ± 0.11 | 3.02 ± 0.18 | 0.51 ± 0.23 | 0.00 ± 0.00 | 6.82 ± 0.11 | 9.90 ± 1.77 | 1.85 ± 0.62 | 7.89 ± 0.31 | 29.47 ± 0.98 | 26.45 ± 0.68 |
| 20° C. | 2.76 ± 0.20 | 3.01 ± 0.32 | 0.30 ± 0.20 | 0.39 ± 0.28 | 6.79 ± 0.41 | 9.96 ± 1.07 | 1.20 ± 0.77 | 6.45 ± 0.87 | 27.81 ± 0.78 | 24.40 ± 0.47 |
| 30° C. | 2.58 ± 0.27 | 2.48 ± 0.37 | 0.31 ± 0.26 | 0.42 ± 0.08 | 6.42 ± 0.36 | 9.42 ± 0.95 | 1.11 ± 0.13 | 6.03 ± 0.96 | 25.89 ± 0.80 | 22.98 ± 0.72 |

Note:
LA—linoleic acid, GLA—γ-linolenic acid, ALA—α-linolenic acid, SDA—stearidonic acid, DGLA—dihomo-γ-linolenic acid, ARA—arachidonic acid, ETA—eicosatetraenoic acid, EPA—eicosapentaenoic acid.
Values are represented as mean ±SD (n = 3)

The total amount of VLCPUFAs was highest when the fungus was grown at 10° C., followed by 20° C., and lowest at 30° C. This fatty acid variation was also reflected in the conversion efficiencies of both Δ6 desaturation and Δ6 elongation at three different temperatures. Without being bound by theory, these results suggest that VLCPUFAs might play an important role in acclimation of the fungus to the temperature shift. When the fungus grew at the lower temperature, the increased VLCPUFAs in membrane lipids would help in maintaining the membrane fluidity and preventing it from the cold damage. The increased long chain unsaturated fatty acids have been previously observed in improving the cold stress in plants (Upchurch 2008; Welti et al., 2002). The change in unsaturated fatty acid levels has also been observed in S. cerevisiae (Nakagawa et al., 2002) where the increased desaturation of cellular fatty acids in cold adaptation is mediated by a transcription factor Mga2p that contributes to the transduction of low-temperature signals for the activation of the Δ9 desaturase. In Synechocystis sp., the signal transduction for the fatty acid desaturation at low temperature is monitored by a “two-component system” composed of a membrane-associated kinase as the signal acceptor (thermosensor) and a response regulator activated upon phosphorylation (Suzuki et al., 2000). The biosynthesis of very long chain unsaturated fatty acids (≧20 C) and long chain unsaturated fatty acids (16-18 C) is different in microbes.

Very long chain ω-3 fatty acids have been shown to have many health benefits. Reconstitution of the VLCPUFA biosynthetic pathway has been attempted in yeast (Beaudoin et al., 2000; Domergue et al., 2003) and plants (Abbadi et al., 2004; Qi et al., 2002; Wu et al., 2005). In yeast, EPA was produced in the presence of ALA using a C. elegans Δ6 elongase, a M. alpina Δ5 desaturase and a borage Δ6 desaturase (Beaudoin et al., 2000). EPA was produced using a different set of three enzymes in presence of ALA (Domergue et al., 2002). The DHA biosynthetic pathway was reconstituted in yeast starting with the Δ6 elongation step (Meyer et al., 2004). In all these cases, an exogenous fatty acid was supplied to the yeast transformants for the production of very long chain ω-3 polyunsaturated fatty acids. The reconstitution of the entire biosynthetic pathway of DGLA, an ω6 VLCPUFA in S. cerevisiae was previously reported using a yeast Kluyveromyces lactis Δ12 desaturase, a rat Δ6 desaturase and a rat Δ6 elongase without supplementation of any foreign fatty acids (Yazawa et al., 2007).

Yeast lacks both Δ12-desaturase and ω-3 desaturase enzymes, thus is unable to produce linoleic acid (LA, 18:2-9, 12) and α-linolenic acid (ALA, 18:3-9,12,15), two precursors for Δ6 desaturation and subsequently Δ6 elongation for the ETA biosynthesis. However, yeast naturally produces substantial amounts of oleic acid (18:1-9), a precursor for LA biosynthesis. Therefore, in one embodiment, the invention may comprise a transformant comprising a heterologous Δ12 desaturase, ω-3 desaturase, Δ6 elongase and Δ6 desaturase, which transformant is able to synthesize ETA without exogenous supplementation of any fatty acids.

In one embodiment, an entire ETA pathway was reconstituted by expressing four genes simultaneously, CoD6, CoE6, CpDes12 for a Δ12 desaturase and CpDesX for an ω-3 desaturase (Meesapyodsuk et al., 2007). The fatty acid analysis of transformants showed that compared with the control, yeast expressing the four genes produced ten new fatty acids. The identity of these fatty acids was confirmed by comparing their retention times as well as their mass spectra to those of standards. 16:2-9,12 and 16:3-9,12,15 are the sequential Δ12 desaturated and Δ15 desaturated products of 16:1-9. 18:2-9,12 (LA) and 18:3-9,12,15 (ALA) are the sequential Δ12 and ω-3 desaturated products of 18:1-9. 18:2-11,14 is the elongated product of 16:2-9,12; 18:3-6,9,12 (GLA) and 18:4-6,9,12,15 (SDA) are two Δ6 desaturated products of LA and ALA. 20:3-11,14,17 is the elongated product of 18:3-9,12,15. 20:3-8,11,14 (DGLA) and 20:4-8,11,14,17 (ETA) are Δ6 elongated products of GLA and SDA. Without being bound by theory, these results indicated that the entire ETA pathways were successfully reconstituted in yeast.

TABLE 2
The fatty acid composition of the yeast where the entire ETA pathway was
reconstituted (the area percent of the total fatty acids, % TFA)

| Fatty acids | % TFA | Conversion efficiency (%) |
|---|---|---|
| 16:0 | 19.08 ± 0.28 | |
| 16:1-9 | 32.99 ± 0.58 | |
| 16:2-9,12 | 6.99 ± 0.24 | 15.36 ± 0.25 |
| 16:3-9,12,15 | 4.50 ± 0.42 | 35.95 ± 0.69 |
| 18:0 | 7.06 ± 0.26 | |
| 18:1-9 | 18.05 ± 0.32 | |
| 18:2-9,12 | 7.67 ± 0.42 | 37.63 ± 1.21 |
| 18:2-11,14 | 1.03 ± 0.02 | 8.21 ± 0.10 |
| 18:3-6,9,12 | 0.13 ± 0.01 | 4.25 ± 0.13 |
| 18:3-9,12,15 | 2.79 ± 0.08 | 27.97 ± 1.18 |
| 18:4-6,9,12,15 | 0.09 ± 0.01 | 6.30 ± 0.31 |
| 20:3-8,11,14 | 0.21 ± 0.01 | 3.97 ± 0.99 |
| 20:3-11,14,17 | 0.12 ± 0.01 | 62.97 ± 0.92 |
| 20:4-8,11,14,17 | 0.11 ± 0.01 | 55.31 ± 5.41 |

New fatty acids in yeast cells are shown in bold.
Values are represented as mean ± SD (n = 3).

While the level of the final product ETA is still low, accounting for about 0.1% of the total fatty acids, it is still conclusive that the entire ETA pathway was successfully reconstituted. Many factors could affect the yield of the final product in a reconstituted metabolic pathway. This may include the activity of the transgene per se, the choice of the host expression system, the activity of a promoter used to control the transgene, and growing condition of transformants.

Puccinia graminis is an obligate fungal pathogen which causes stem rust of small cereal crops such as oat, wheat and barley (Leonard et al., 2005). The PgDesX cDNA encoding ω-3 desaturase from spore of P. graminis was cloned. The total RNAs isolated from P. graminis was used to synthesize first-strand cDNA. The cDNA was then used as a template for the PCR reaction with two gene specific primers designed from the predicted full length cDNA obtained from P. graminis f. sp. tritici sequence database (Broad Institute).

To determine the function of PgDesX, the coding region of cDNA was cloned into the yeast expression vector pYES2.1 under control of GAL1 promoter, and the recombinant plasmids were then introduced into S. cerevisiae INVSc1. The analysis of transformants showed that, compared with the yeast negative control (pYES2.1/INVSc1), PgDesX/INVSc1 expressing PgDesX produced two new peaks, which were identified as 16:2-9c, 12c and 16:3-9c, 12c, 15.

To study the substrate specificity of PgDesX, PgDesX/INVSc1 were grown in minimal medium supplemented separately with 18:2-9c, 12c, 18:3-6c, 9c, 12c, 20:3-8c, 11c, 14c, and 20:4-5c, 8c, 11c, 14c. The results showed that CpDesX possessed a substrate preference for 18 C fatty acids, linoleic acid in particular. The highest conversion efficiency (products as a percentage of the sum of substrates and products) was observed on 18:2-9c, 12c (76%), followed by 18:3-6c, 9c, 12c (50%), and 20:4-5c, 8c, 11c, 14c (38%), and 20:3-8c, 11c, 14c (25%), indicating that PgDesX is a new ω-3 desaturase able to converting ω-6 fatty acids with 16 C-20 C chains to their corresponding ω-3 fatty acids. The unique property of this enzyme would have potentials in production of ω-3 fatty acids in heterologous systems, especially in oilseed crops for nutraceutical uses.

Exemplary embodiments of the present invention are described in the following Examples, which are set forth to aid in the understanding of the invention, and should not be construed to limit in any way the scope of the invention as defined in the claims which follow thereafter.

### Example 1

### The Fungus Strain and Growth Conditions

Five entomopathogenic fungi Conidiobolus thromboids (ARSEF120), Conidiobolus obscurus (ARSEF74 kindly provided by Dr. Richard Humber, Robert W. Holley Center for Agriculture and Health, Ithaca, N.Y., USA), Entomophora conglomenta (ARSEF2273), Batkoa spp. (ARSEF3131), and Batkoa gigantea (ARSEF215) were grown in a quarter strength of Sabourad dextrose media (10 g/L) containing 5 g/L dextrose, 2.5 g/L bactopeptone and 2.5 g/L yeast extract, and grown at room temperature (22° C.) for 10 days with shaking at 180 rpm. The cells were harvested by vacuum filtration and washed twice with 20 mL of sterile water. After freeze-drying, the cells were stored at −80° C. until use for fatty acid analysis and gene cloning.

### Example 2

### Gene Expression Analysis Under Different Temperature Conditions

C. obscurus was first grown in 200 mL culture at 20° C. for 5 days and the cells were then harvested and resuspended in 50 mL of the fresh medium. Each 5 mL aliquot of the suspension was added with 10 mL of the fresh medium. Samples were then incubated at 10° C., 20° C. and 30° C. for 24 hrs. After that, the cells were collected by vacuum filtration and washed twice with distilled water. Total RNAs from the fungal samples were extracted using TRIzol reagent (Invitrogen, Burlington, ON, Canada). One μg of total RNA was treated with DNase I and used for cDNA synthesis using SuperScript III RT-PCR system (Invitrogen, Burlington, ON, Canada) in 20 μl reaction with random primers. The half μl of first-strand reaction was then used as a template for 25-μl multiplex PCR reaction using Taq DNA Polymerase (UBI Life Sciences, Saskatoon, SK, Canada). The specific primers LT53 (5′-ATCTTGGTGCGCATATAGCATGTGGTTC-3′; SEQ ID NO: 15) and LT59 (5′-GGATCCTTAATCCTGTTTAGGAGGTTCAG-3′; SEQ ID NO: 16) were used to generate a 565 bp CoD6 cDNA, primers LT60 (5′-GCGGCCGCATTATGGCCTCAGCAGTTTAC-3′; SEQ ID NO: 17) and LT57 (5′-AACCACCAGACGCCAAAGATGGAGCAG-3′; SEQ ID NO: 18) were used to generated a 608 bp CoE6 cDNA. The 18S rRNA primer-competitor mix (Universal 18S internal standard kit; Ambion, Applied Biosystems, Streetsville, ON, Canada) was used for amplification of the internal control in multiplex PCR experiments. The PCR conditions for both multiplex PCR reactions were 25 cycles of 95° C. for 30 sec, 55° C. for 30 sec and 72° C. for 40 sec. A 10-μl aliquot of both reactions was then analyzed on a 1.5% agarose gel and amplified products were quantified by the gel-documentation system (Alpha Imager, HP System, Santa Clara, Calif., USA).

### Example 3

### Cloning Putative Delta-6 Desaturase and Delta-6 Elongase Genes from C. obscurus

Total RNA was extracted from the fungal biomass of C. obscurus using Trizol reagent and 5 μg of total RNA was used to synthesize first-strand cDNA using the SuperScript III first-strand synthesis system (Invitrogen, Burlington, ON, Canada). To clone the putative Δ6 desaturase gene (CoD6), 2 μL of the first strand cDNA was used as a template for PCR amplification with the degenerate oligonucleotide primers LT14 (5′-YTGNARNCCNCCRRGRAACCA-3′; SEQ ID NO: 19) and LT16 (5′-ATHGMNRANCAYCCNGGNGG-3′; SEQ ID NO: 20) that were designed based on conserved amino acid regions of Δ6 desaturase enzymes from Mucor rouxii (Laoteng et al., 2000), Mortierella alpina (Huang et al., 1999), Rhizopus stolonifer (Zhang et al., 2004), Thamnidium elegans (Wang et al., 2007), Physcomitrella patens (Girke et al., 1998), Pythium irregulare (Hong et al., 2002), Cunninghamella echinulata (Fakas et al., 2006) and Caenorhabditis elegans (Napier et al., 1998). The forward primer LT14 and the reverse primer LT16 correspond to the conserved regions W-F-H-G-G-L-Q (SEQ ID NO: 21) and I-(A/E)-(D/K)-H-P-G-G (SEQ ID NO: 22), respectively. Amplified products with the expected size of approximately ˜1000 bp were gel purified, cloned into the pCR4-TOPO vector (Invitrogen, Burlington, ON, Canada) and sequenced. The 5′ and 3′ ends of the CoD6 cDNA were obtained by RACE using a Marathon cDNA amplification kit (Clontech, Mountain View, Calif., USA) following the manufacturer's recommendations. Primers LT50 (5′-AGAGTTCCATAGCGTTCTCGGACCAGGC-3′; SEQ ID NO: 23) LT51 (5′-TGCCATCCACATTGTTGAAAGAAGAGTCC-3′; SEQ ID NO: 24) were used to obtain the 5′ end, while primers LT52 (5′-TGGGTTGGGGGTCACTTCTTTGGAGC-3′; SEQ ID NO: 25) and LT53 (5′-ATCTTGGTGCGCATATAGCATGTGGTTC-3′; SEQ ID NO: 26) were used to obtain the 3′ end. The full-length cDNA sequence, including the 5′ and 3′ untranslated regions as well as the coding region, was retrieved by RT-PCR using Phusion polymerase (New England Biolabs, Pickering, ON, Canada) with the specific primers LT58 (5′-GGATCCATCATGGCACCTCTTACTAAC-3′; SEQ ID NO: 27) and LT59 (5′-GGATCCTTAATCCTGTTTAGGAGGTTCAG-3′; SEQ ID NO: 28).

To isolate putative Δ6 elongase gene (CoE6), the degenerate oligonucleotide primers LT5 (5′-TTYTTNCAYGTNTAYCAYCA-3′; SEQ ID NO: 29) and LT6 (5′-ARRAARTARTANCCRTACAT-3′; SEQ ID NO: 30) were designed which correspond to the conserved amino acid regions F-L-H-V-Y-H-H (SEQ ID NO: 31) and M-Y-T-Y-Y-F-L-S (SEQ ID NO: 32) of Δ6 elongase enzymes from M. alpina (Parker-Barnes et al., 2000), Thalassiosira pseudonana (Meyer et al., 2004), Phaeodactylum tricornutum (Domergue et al., 2002), Ostreococcus tauri (Meyer et al., 2004), P. patens (Zank et al., 2000) and Oncorhynchus mykiss (Meyer et al., 2004). Amplified products with the expected size of approximately 150 bp from degenerate RT-PCR were gel purified, cloned into the pCR4-TOPO vector and sequenced. The 5′ and 3′ ends of the CoE6 cDNA were obtained by RACE using a Marathon cDNA amplification kit following the manufacturer's recommendations. Primers LT56 (5′-ATCACGTGGATGTAGGAGTTAAGGGCAG-3′; SEQ ID NO: 33) and LT57 (5′-AACCACCAGACGCCAAAGATGGAGCAG-3′; SEQ ID NO: 34) were used to obtain the 5′ end while primers LT54 (5′-TCTTCCACGTCTACCACCACTGCTCC-3′; SEQ ID NO: 35) and LT55 (5′-TCAGCTGCCCTTAACTCCTACATCCACG-3′; SEQ ID NO: 36) were used to obtain the 3′ end. The full-length sequence including the 5′ and 3′ untranslated regions as well as the coding region was retrieved by RT-PCR using Phusion polymerase with the specific primers LT60 (5′-GCGGCCGCATTATGGCCTCAGCAGTTTAC-3′; SEQ ID NO: 37) and LT61 (5′-GCGGCCGCTTAGTTGCGCTTTTTGCCATAG-3′; SEQ ID NO: 38). The nucleotide and amino acid sequences for CoD6 and CoE6 have been deposited in GenBank under accession numbers HQ656805 and HQ656806, respectively.

### Example 4

### Heterologous Expression of CoD6 and CoE6 in Yeast

To express the genes in yeast, the open reading frames were inserted into the vector pYES2.1/V5-His-TOPO behind the GAL1 promoter. The recombinant plasmids were introduced into the yeast host S. cerevisiae INVSc1 using the lithium acetate transformation method (Gietz et al., 1992). The yeast transformants were first grown in a synthetic yeast medium containing 2% glucose, 0.67% bacto-yeast nitrogen base lacking uracil at 28° C. for 2 days. The cultures were then washed twice with distilled water and resuspended in 10 mL of the induction medium (the synthetic yeast medium containing 2% galactose instead of 2% glucose) supplemented with or without 250 μM fatty acid substrate in the presence of 0.1% tergitol. The induced cultures were grown at 20° C. for 2 days.

### Example 5

### Fatty Acid Analysis

The fatty acids of yeast cells were directly transmethylated with 2 mL of 3N methanolic HCl at 80° C. for 1 hour. After the transmethylation process, the sample was cooled down at room temperature before adding 1 mL of 0.9% NaCl and 2 mL of hexane. The sample was then mixed and centrifuged at 2,400 rpm for 5 minutes for phase separation. Hexane phase containing fatty acid methyl esters (FAMEs) were removed and dried under N₂. After drying, the sample was resuspended in 400 μL of hexane and placed in a GC auto-sampler vial for GC analysis. Two μl, of the total FAMEs sample was analyzed on an Agilent 6890N gas chromatograph equipped with a DB-23 column with 0.25-μm-film thickness (J&W Scientific). The column temperature was maintained at 160° C. for 1 min, and then raised to 240° C. at a rate of 4° C./min (Reed et al., 2000). The areas of chromatographic peaks were calculated for relative amounts of FAMEs. GC-MS analysis was accomplished using an Agilent 5973 mass selective detector coupled to an Agilent 6890N gas chromatograph with the same column and conditions as described above. The mass selective detector was run under standard electron impact conditions (70 eV), scanning an effective m/z range of 40-700 at 2.26 scans/sec.

For fatty acid analysis of C. obscurus, the fungus was grown in 40 mL of quarter strength of the Sabouraud medium (SAB) at the room temperature (22° C.) for 10 days. The fungal cells was harvested through vacuum filtration and washed twice with 20 mL of distilled water. The sample was then mixed with 7 mL of 2:1 chloroform:methanol (v/v) mixture and homogenized for two minutes. The homogenized solution was centrifuged at 2400 rpm for 5 minutes. The bottom lipid layer was carefully transferred into a new tube and dried under a N₂ stream. The dried lipid was resuspended in an appropriate volume of chloroform to achieve a final concentration of 20 μg/μL. To fractionate the different lipid classes, the total lipid extract was resolved on silica G-25 thin layer chromatography and developed with hexane/diethyl ether/acetic acid (70:30:1, v/v/v) for neutral lipids and with chloroform/methanol/acetic acid/water (100:40:12:2, v/v/v/v) for phospholipids, respectively.

Once the solvent front reached approximately two centimeters from the top of the plate, the developed plate was air dried and sprayed with the lipid staining solution (5 mg primuline in 100 mL of 80:20 acetone:water [v/v]). Lipid staining was observed under an UV transluminator (Aitzetmüller et al., 1992). In reference to the lipid standard, the corresponding spots to each lipid class were then scratched off the silica plate and directly transmethylated as described above.

### Example 6

### Reconstruction of the ETA Pathway in Yeast

To reconstitute the ETA pathway, the CoD6 gene flanked with BamHI sites was first inserted in the yeast pESC-HIS vector (Stratagene) behind the GAL1 promoter, while CoE6 was cloned in the NotI site of the vector behind the GAL10 promoter. The yeast co-expression vector pESC-URA (Stratagene) was used to clone CpDes12 and CpDesX where CpDes12 was under the control of GAL1 promoter and CpDesX was under the control of the GAL10 promoter. To facilitate the CpDesX cloning process, BglII restriction site was incorporated at the 5′ ends of the forward and reverse primers, LT48 (5′-GAAGATCTTCGAAATGGCTAACAAATCTCC-3′; SEQ ID NO: 39) and LT49 (5′-GAAGATCTTCCTAGCCGTGTGTGTGGAC-3′; SEQ ID NO: 40). These primers were then used to amplify the full-length CpDesX, the amplified product was digested with BglII and inserted into its respective digested site within the pESC-URA vector. For cloning CpDes12, the plasmid containing the gene was cut with the restriction enzymes BamHI and EcoRI, and ligated into the sites of pESC-URA. The two recombinant plasmids expressing the four genes were co-transformed into S. cerevisiae INVSc1 using the lithium acetate transformation method (Gietz et al., 1992), the yeast transformants were selected on a selection medium lacking histidine and uracil, and containing 2% glucose. To assess the expression of the reconstituted ETA pathway, the transgenic yeast cells were first grown in a synthetic yeast medium lacking histidine and uracil and containing 2% glucose and 0.67% bacto-yeast nitrogen base at 28° C. for 2 days. The cultures were then washed twice with distilled water and resuspended with the induction medium (the synthetic yeast medium containing 2% galactose). The induced cultures were incubated at 15° C. for 2 days, then at 20° C. for 2 days. Following the induction, the yeast cells were harvested by centrifugation at 2400 rpm and washed once with 15 mL of 0.1% tergitol and twice with 10 mL of distilled water.

### REFERENCES

The following references are incorporated herein by reference (where permitted) as if reproduced in their entirety. All references are indicative of the level of skill of those skilled in the art to which this invention pertains.
Abbadi A, Domergue F, Bauer J, Napier J A, Welti R, Zahringer U, Cirpus P, Heinz E (2004) Biosynthesis of very-long-chain polyunsaturated fatty acids in transgenic oilseeds: Constraints on their accumulation. Plant Cell 16: 2734-2748.
Aitzetmüller K, Xin Y N, Werner G, Grönheim M (1992) High-performance liquid-chromatographic investigations of stillingia oil. J Chromatogr 603:165-173.
Beaudoin F, Michaelson L V, Hey S J, Lewis M. J, Shewry P R, Sayanova O, Napier J A (2000) Heterologous reconstitution in yeast of the polyunsaturated fatty acid biosynthetic pathway. P Natl Acad Sci USA 97: 6421-6426.
Cheng B, Wu G, Vrinten P, Falk K, Bauer J, Qiu X (2010) Towards the production of high levels of eicosapentaenoic acid (EPA) in transgenic plants: the effects of different host species, genes and promoters. Transgenic Res 19: 221-229.
Domergue F, Abbadi A, Ott C, Zank T K, ahringer U, Heinz E (2003) Acyl carriers used as substrates by the desaturases and elongases involved in very long-chain polyunsaturated fatty acids biosynthesis reconstituted in yeast. J Biol Chem 278:35115-35126.
Domergue F, Abbadi, A, ahringer U, Moreau H, Heinz E (2005) In vivo characterization of the first acyl-CoA Δ6-desaturase from a member of the plant kingdom, the microalga Ostreococcus tauri. Biochem J 389: 483-490.
Domergue F, Lerchl J, ahringer U, Heinz E (2002) Cloning and functional characterization of Phaeodactylum tricornutum front-end desaturases involved in eicosapentaenoic acid biosynthesis. Eur Biochem 269: 4105-4113.
Fakas S, Papanikolaou S, Galiotou-Panayotou M, Komaitis M, Aggelis G (2006) Lipids of Cunninghamella echinulata with emphasis to γ-linolenic acid distribution among lipid classes. Appl Microbiol Biot 73: 676-683.
Feng M G, Johnson J B, Kish L P (1990) Survey of entomopathogenic fungi naturally infecting cereal aphids (Homoptera: Aphidiae) of irrigated grain crops in southwestern Idaho. Environ Entomol 19: 1534-1542.
Gietz D, St Jean A, Woods R A, Schiestl R H (1992) Improved method for high-efficiency transformation of intact yeast-cells. Nucleic Acids Res 20: 1425-1425.
Girke T, Schmidt H, Zahringer U, Reski R, Heinz E (1998) Identification of a novel Δ6-acyl-group desaturase by targeted gene disruption in Physcomitrella patens. Plant Journal 15: 39-48.
Hoffmann M, Wagner M, Abbadi A, Fulda M, Feussner I (2008) Metabolic engineering of omega 3-very long chain polyunsaturated fatty acid production by an exclusively acyl-CoA-dependent pathway. Biol Chem 283: 22352-22362.
Hong H, Datla N, Reed D W, Covello P S, MacKenzie S L, Qiu X (2002) High-level production of γ-linolenic acid in Brassica juncea using a Δ6 desaturase from Pythium irregulare. Plant Physiol 129: 354-362.
Huang Y S, Chaudhary S, Thurmond J M, Bobik E G, Yuan L, Chan G M, Kirchner S J, Mukerji P, Knutzon D S (1999) Cloning of Δ12 and Δ6-desaturases from Mortierella alpina and recombinant production of γ-linolenic acid in Saccharomyces cerevisiae. Lipids 34:649-659.
Kajikawa M, Yamato K T, Kohzu Y, Nojiri M, Sakuradani E, Shimizu S, Sakai Y, Fukuzawa H, Ohyama K (2004) Isolation and characterization of Δ6-desaturase, an ELO-like enzyme and Δ5-desaturase from the liverwort Marchantia polymorpha and production of arachidonic and eicosapentaenoic acids in the methylotrophic yeast Pichia pastoris. Plant Mol Biol 54: 335-352.
Kaijikawa M, Matsui K, Ochiai M, Tanaka Y, Kita Y, Ishimoto M, Kohzu Y, Shoji S, Yamato K T, Ohyama K, Fukuzawa H, Kohchi T (2008) Production of arachidonic and eicosapentaenoic acids in plants using bryophyte fatty acid Δ6 desaturase, Δ6 elongase, and Δ5 desaturase genes. Biosci Biotechnol Biochem 72: 435-444.
Khunyoshyeng S, Cheevadhanarak S, Rachdawong S, Tanticharoen M (2002) Differential expression of desaturases and changes in fatty acid composition during sporangiospore germination and development in Mucor rouxii. Fungal Genet Biol 37: 13-21.
Laoteng K, Mannontarat R, Tanticharoen M, Cheevadhanarak S (2000) Δ6)-Desaturase of Mucor rouxii with high similarity to plant Δ6-desaturase and its heterologous expression in Saccharomyces cerevisiae. Biochem Bioph Res Co 279: 17-22.
Lopez Alonso D, Garcia-Maroto F, Rodriguez-Ruiz J, Gamido J A, Vilches M A (2003) Evolution of the membrane-bound fatty acid desaturases. Biochem Syst Ecol 31: 1111-1124.
Mackichan J, Thomsen L, Kerwin J, Latge J P, Beauvais A (1995) Unsaturated fatty-acids are the active molecules of glucan-synthase-inhibitory fraction isolated from entomophthoralean protoplasts. Microbiol-UK 141: 2757-2762.
Meesapyodsuk D, Reed D W, Covello P S, Qiu X (2007) Primary structure, regioselectivity, and evolution of the membrane-bound fatty acid desaturases of Claviceps purpurea. J Biol Chem 282: 20191-20199.
Meyer A, Kirsch H, Domergue F, Abbadi A, Sperling P, Bauer J, Cirpus P, Zank T K, Moreau H, Roscoe T J, Zahringer U, Heinz E (2004) Novel fatty acid elongases and their use for the reconstitution of docosahexaenoic acid biosynthesis. J Lipid Res 45: 1899-1909.
Michinaka Y, Aki T, Shimauchi T, Nakajima T, Kawamoto S, Shigeta S, Suzuki O, Ono K (2003) Differential response to low temperature of two Δ6 fatty acid desaturases from Mucor circinelloides. Appl Microbiol Biot 62: 362-368.
Milner R J, Soper R S (1981) Bioassay of Entomophthora against the spotted alfalfa aphid Therioaphis-trifolii f-maculata. J Invertebr Pathol 37: 168-173.
Nakagawa T, Yamada K, Miyaji T, Tomizuka N (2002) Cold-active pectinolytic activity of psychrophilic-basidiomycetous yeast Cystofilobasidium capitatum strain PPY-1. J Biosci Bioeng 94:175-177.
Napier J A (2006) The production of n-3 long-chain polyunsaturated fatty acids in transgenic plants. Eur Lipid Sci Tech 108: 965-972.
Napier J A, Hey S J, Lacey D J, Shewry P R (1998) Identification of a Caenorhabditis elegans Δ6-fatty-acid-desaturase by heterologous expression in Saccharomyces cerevisiae. Biochem J 330: 611-614.
Nukina M, Sassa T, Ikeda M, Takahashi K, Toyota S (1981) Linoleic acid enhances perithecial production in Neurospora crassa. Agr Biol Chem Tokyo 45: 2371-2373
Parker-Barnes J M, Das T, Bobik E, Leonard A E, Thurmond J M, Chaung L T, Huang Y S, Mukerji P (2000) Identification and characterization of an enzyme involved in the elongation of n-6 and n-3 polyunsaturated fatty acids. P Natl Acad Sci USA 97: 8284-8289.

Qi B, Beaudoin F, Fraser T, Stobart A K, Napier J A, Lazarus C M (2002) Identification of a cDNA encoding a novel C18-A9 polyunsaturated fatty acid-specific elongating activity from the docosahexaenoic acid (DHA)-producing microalga, Isochrysis galbana. FEBS Lett 510: 159-165.

Ratledge C, Wynn J P (2002) The biochemistry and molecular biology of lipid accumulation in oleaginous microorganisms. Adv Appl Microbiol, Vol 51, pp. 1-51.
Reed D W, Schafer U A, Covello P S (2000) Characterization of the Brassica napus extraplastidial linoleate desaturase by expression in Saccharomyces cerevisiae. Plant Physiol 122: 715-720.
Scorsetti A C, Humber R A, Garcia J J, Lastra CCL (2007) Natural occurrence of entomopathogenic fungi (Zygomycetes: Entomophthorales) of aphid (Hemiptera: Aphididae) pests of horticultural crops in Argentina. Biocontrol 52: 641-655.
Steinkraus D C, Tugwell N P (1997) Beauveria bassiana (Deuteromycotina: Moniliales) effects on Lygus lineolaris (Hemiptera: Miridae). J Entomol Sci 32: 79-90.
Suzuki I, Los D A, Murata N (2000) Perception and transduction of low-temperature signals to induce desaturation of fatty acids. Biochem Soc T 28: 628-630.
Tanada Y, Kaya H K (1993) Insect Pathology. Academic Press Limited, San Diego, Calif., pp 336-356.
Tyrrell D (1967) Fatty acid composition of 17 entomophthora isolates. Canadian J Microbiol 13: 755-760.
Upchurch R G (2008) Fatty acid unsaturation, mobilization, and regulation in the response of plants to stress. Biotechnol Lett 30: 967-977.

Venegas-Caleron M, Sayanova O, Napier J A (2010) An alternative to fish oils: Metabolic engineering of oil-seed crops to produce omega-3 long chain polyunsaturated fatty acids. Prog Lipid Res 49:108-119.

Wang D P, Li M C, Wei D S, Cai Y, Zhang Y H, Xing L J (2007) Identification and functional characterization of the Δ6-fatty acid desaturase gene from Thamnidium elegans. J Eukaryot Microbiol 54: 110-117.
Welti R, Li W Q, Li M Y, Sang Y M, Biesiada H, Zhou H E, Rajashekar C B, Williams T D, Wang X M (2002) Profiling membrane lipids in plant stress responses—Role of phospholipase D ain freezing-induced lipid changes in Arabidopsis. J Biol Chem 277: 31994-32002.
Wu G H, Truksa M, Datla N, Vrinten P, Bauer J, Zank T, Cirpus P, Heinz E, Qiu X (2005) Stepwise engineering to produce high yields of very long-chain polyunsaturated fatty acids in plants. Nat Biotechnol 23: 1013-1017.
Yazawa H, Iwahashi H, Kamisaka Y, Kimura K, Aki T, Ono K, Uemura H (2007) Heterologous production of dihomo-γ-linolenic acid in Saccharomyces cerevisiae. Appl Environ Microb 73: 6965-6971.
Zank T K, Zäluinger U, Lerchl J, Heinz E (2000) Cloning and functional expression of the first plant fatty acid elongase specific for Δ6 polyunsaturated fatty acids. Biochem Soc T 28: 654-658.
Zhang Q, Li M C, Ma H T, Sun Y, Xing L J (2004) Identification and characterization of a novel A6 fatty acid desaturase gene from Rhizopus arrhizus. FEBS Lett 556: 81-85.

What is claimed is:

1. An isolated polynucleotide encoding a polypeptide having Δ5, Δ6, or ω-3 desaturase activity or Δ6 elongase activity and comprising:
(a) an amino acid sequence selected from SEQ ID NO: 2, 4, 6, 8 or 10; or
(b) an amino acid sequence having at least 85% sequence identity with one of SEQ ID NO: 2, 4, 6, 8 or 10.

2. The isolated polynucleotide of claim 1, wherein the polynucleotide encodes a polypeptide having Δ5 desaturase activity and comprising the amino acid sequence of SEQ ID NO: 2 or an amino acid sequence having Δ5 desaturase activity and having at least 85% sequence identity therewith.

3. The isolated polynucleotide of claim 1, wherein the polynucleotide encodes a polypeptide having Δ6 desaturase activity and comprising the amino acid sequence of SEQ ID NO: 4 or an amino acid sequence having Δ6 desaturase activity and having at least 85% sequence identity therewith.

4. The isolated polynucleotide of claim 1, wherein the polynucleotide encodes a polypeptide having ω-3 desaturase activity and comprising the amino acid sequence of SEQ ID NO: 10 or an amino acid sequence having ω-3 desaturase activity and having at least 85% sequence identity therewith.

5. The isolated polynucleotide of claim 1, wherein the polynucleotide encodes a polypeptide having Δ6 elongase activity and comprising the amino acid sequence of SEQ ID NO: 6 or 8, or an amino acid sequence having Δ6 elongase activity and having at least 85% sequence identity with one of SEQ ID NO: 6 or 8.

6. The isolated polynucleotide of claim 1, wherein the polynucleotide comprises the nucleotide sequence of SEQ ID NO: 1, 3, 5, 7 or 9.

7. The isolated polynucleotide of claim 1, wherein the encoded polypeptide comprises an amino acid sequence having at least 90%, at least 95%, at least 96%, at least 97%, at least 98%, or at least 99% sequence identity to one of SEQ ID NO: 2, 4, 6, 8 or 10.

8. The isolated polynucleotide of claim 1, wherein the polynucleotide is derived from Conidiobolus obscurus, Conidiobolus thromboids, or Puccinia graminis.

9. A polynucleotide construct or vector comprising a polynucleotide of claim 1 operably linked to a promoter expressible in bacterial, yeast, fungal, mammalian or plant cells.

10. A microbial cell comprising a polynucleotide construct or vector of claim 9.

11. The microbial cell of claim 10, wherein the cell is Saccharomyces cerevisiae, Aspergillus, Pichia pastoris, Escherichia coli or Bacillus subtilis.

12. The microbial cell of claim 11 which comprises a heterologous eicosatetraenoic acid biosynthetic pathway comprising a Δ6 desaturase, a Δ6 elongase, a Δ12 desaturase and an ω3 desaturase.

13. The microbial cell of claim 12 which is S. cerevisiae and which comprises CoD6 and CoE6 from C. obscurus, CpDes12 and CpDesX from Claviceps purpurea.

14. A transgenic plant, plant cell, plant seed, callus, plant embryo, microspore-derived embryo, or microspore, comprising a polynucleotide construct or vector of claim 9.

15. The transgenic plant, plant cell, plant seed, callus, plant embryo, microspore-derived embryo, or microspore of claim 14, which is selected from a linseed, canola, peanut, safflower, flax, oats, wheat, triticale, barley, corn, and legume plant, plant cell, plant seed, callus, plant embryo, or microspore-derived embryo or microspore.

16. A method for producing a polyunsaturated fatty acid comprising the steps of:
a) constructing a vector comprising one or more polynucleotides of claim 1;
b) transforming the vector into a host cell under conditions sufficient for expression of a Δ5, Δ6 or ω-3 desaturase or a Δ6 elongase encoded by the polynucleotides; and
c) exposing the Δ5, Δ6, or ω-3 desaturase or A6 elongase to a fatty acid substrate, wherein the substrate is converted by the Δ5, Δ6, or ω-3 desaturase or A6 elongase into a desired polyunsaturated fatty acid product.

17. The method of claim 16, wherein the fatty acid substrate comprises one or more of linoleic acid, γ-linolenic acid, α-linolenic acid, stearidonic acid, dihomo-γ-linolenic acid, arachidonic acid, eicosatetraenoic acid, and eicosapentaenoic acid.

18. The method of claim 17, wherein the polypeptide comprises a Δ6 desaturase, and the fatty acid substrate comprises linoleic acid or α-linolenic acid.

19. The method of claim 17, wherein the polypeptide comprises a Δ6 elongase, and the fatty acid substrate comprises γ-linolenic acid, stearidonic acid, arachidonic acid or eicosapentaenoic acid.

20. The method of claim 17, wherein the polypeptide comprises an ω-3 desaturase, and the fatty acid substrate comprises linoleic acid, γ-linoleic acid, dihomo gamma-linoleic acid and arachidonic acid.


SEQUENCE LISTING (40 sequences)

SEQ ID NO: 1 - DNA, 1418 bp, Coniodiobolus obscurus
atggcacctcgctctgaagttttagtcaatccgagtgaagccaacgacgctcaaggaaatttagaccctattactgggaagcagctcaagagtataagctgggaggaattggctcaacataacactagccattcttgttggatagcagttcgaggaaaagtttacgatgttactaaatttctaggacgacaccctggaggaatggatacccttttgtatggagcaggacgggacgccactttagtagtagagacatatcacgacttggacagtttaacagtactactcaataacaagtttttgattggacagctggggtcagatgagcttccaaccttccctttacccaacccatttcaccgtcaacttaaagataaggtcaaggcacactttaaaagcggaactgatcccaagaatccagggctttgggtgatgctgcgatacttgagtatttatggactagttttggggagttgggccgcaacccattctggggtaggaatgtggtctcagattgggttaagtttaattctgggatttggttgcgctttgattggactgcacccacttcatgacgctagtcattttgccataactcactctcccacagtatggaagtggctaggagctactcatgattttttaaatggaacgtcatacttggtgtgggtttatcagcatatgctgggccaccacccttacacaaacattccaggggctgatcctgatattgctactaatgaccctgacttgcggcgtattcaaccctaccagacttggtattctcgctacttaaaccaggagatgtatgtgcctctcctttacggagctttagcctggaagactagggtacaggatattatgattttatacatcgacaaatcaaatgacggaattcgtgttaatcctccaactctataccacaatcttgtgttctggttaggaaaactattctttgttttttatcgtattttattgcctatttatttgtaggaacacgccaggctctagctaatctaatcatctctgatttggtgagctcatattggctagctttgaccttccaagtcaatcatgttgtagacaatgttgattttattcctcttccccctgctgatgctcagagtaaggaaattgatatggattgggctgagatgcaacttagaaccactcaggattatgctcacgaagatactctatggacttgggctacaggcggccttaattaccaagctgttcaccacgtattccctaatattcatcaggctcactaccctgcccttgctccattaatccagagtttatctaaagaacacaatgttccatttatttgtcaggatacatttatggatgcattctggggacacatcaaccacctcaagataatgggcagatcccccgattccaaggagaattaa

SEQ ID NO: 2 - protein, 471 aa, Coniodiobolus obscurus
MAPRSEVLVNPSEANDAQGNLDPITGKQLKSISWEELAQHNTSHSCWIAVRGKVYDVTKFLRHPGGMDTLLYGAGRDATLVVETYHDLDSLTVLLNNKFLIGQLGSDELPTFPLPNPFHRQLKDKVKAHFKSGTDPKNPGLWVMLRYLSIYGLVLGSWAATHSGVGMWSQIGLSLILGFGCALIGLHPLHDASHFAITHSPTVWKWLGATHDFLNGTSYLVWVYQHMLGHHPYTNIPGADPDIATNDPDLRRIQPYQTWYSRYLNQEMYVPLLYGALAWKTRVQDIMILYIDKSNDGIRVNPPTLYHNLVFWLGKLFFVFYRILLPIYLVGTRQALANLIISDLVSSYWLALTFQVNHVVDNVDFIPLPPADAQSKEIDMDWAEMQLRTTQDYAHEDTLWTWATGGLNYQAVHHVFPNIHQAHYPALAPLIQSLSKEHNVPFICQDTFMDAFWGHINHLKIMGRSPDSKEN

SEQ ID NO: 3 - DNA, 1362 bp, Coniodiobolus obscurus
ggatccatcatggcacctcttactaacgaaatccttgctcctcttcagaagaagaaactgtatacaataatcgataataaggcatacgatgtgaccgactttgtgaatgagcacccaggaggtcctgttattatgactcaattggggatcgatgcaactgatgcttttcattgttttcatcccccatctatccaggaaattttgtctgattactatgatgaagagctaaccaaacagttaggccttagagatcgtgaagatactaagttcatcaaagagattcgttctttgaaggaagagtttgttaaggaagggttatttgaggccaacctattattctatggttttatgggagttttcaacctatcaatctttggaacttcagttgctttactcgccaatttcggcgatagcgtactcgcagtgctggtctctgctggacttttaggactcttctttcaacaatgtggatggcacgctcatgagtacctgcatcatcaagtattcaagaatagaaccttcaataactgggttgggggtcacttctttggagcactctgccaaggattttctgcgtcatggtggaaagacaagcataacactcaccatgctgcacctaatgtttactctcacgacccagatatcgacactcatcccttcttggcctggtccgagaacgctatggaactctacgctgagttaaatgaccaagaactcggatctcatcttaagaaatttatgttgcataatcagcctatcctattcttcccccttctcgccattgctcgactatcttggtgcgcatatagcatgtggttcgctattagcaatggccaacttggagatcctaatagaatgtacatccctattcatttctcagagcctctctgtctaataattcactggataatctatttctggatagtacttactctacccgctacttggtcactctccctcttattttttattatatcgcagattacttgtggtgtattattagcttccgtttttaccctaaatcacaacggaatgaaagtgtactcaaaagaggaggcagataagatggatttttactcccttcaggcggaaactggacgagatgtacacccttcctattttatgacttggttctgtggtggcttgaactatcagattgagcatcaccttttcccaactcttccacgtcataactttcaaaggattcaatctagagtgaaagctctgcttaataagtataacataacctatcatgttactgggtttactgaaggaactatggaagttttaaaccgattggatcgggtagcaagatccattgagcaaggattgtctgaacctcctaaacaggattaaggatcc

SEQ ID NO: 4 - protein, 448 aa, Coniodiobolus obscurus
MAPLTNEILAPLQKKKLYTIIDNKAYDVTDFVNEHPGGPVIMTQLGIDATDAFHCFHPPSIQEILSDYYDEELTKQLGLRDREDTKFIKEIRSLKEEFVKEGLFEANLLFYGFMGVFNLSIFGTSVALLANFGDSVLAVLVSAGLLGLFFQQCGWHAHEYLHHQVFKNRTFNNWVGGHFFGALCQGFSASWWKDKHNTHHAAPNVYSHDPDIDTHPFLAWSENAMELYAELNDQELGSHLKKFMLHNQPILFFPLLAIARLSWCAYSMWFAISNGQLGDPNRMYIPIHFSEPLCLIIHWIIYFWIVLTLPATWSLSLLFFIISQITCGVLLASVFTLNHNGMKVYSKEEADKMDFYSLQAETGRDVHPSYFMTWFCGGLNYQIEHHLFPTLPRHNFQRIQSRVKALLNKYNITYHVTGFTEGTMEVLNRLDRVARSIEQGLSEPPKQD

SEQ ID NO: 5 - DNA, 1003 bp, Coniodiobolus obscurus
gcggccgcattatggcctcagcagtttacgagaaggcagcaagcggcatggtgccagctgcctattatgagaaaccagccgatctcatcatcgagtatgtgggcagaggattaaattatgcggccccacttacccaagcagtcgagggggcactcatcaaagccatgcctgaagcatactccaccgtgaccaactatcttgcaacgacccgatctcccctcagcgaggggttccccctgatgaacccggtccaggttctcctagttatggtgtcctacctcactattgtgtttgttggcaaggccatcatgtccaacttcacgcgtattgaggccaagacgttctccttgttccataacttcgccatggtgtccatctctgcttacatgtgctatggcgtggttgttcaggcgctcgctgataagtatactctgttcactaaccctggcgacaataccgctactggctaccccatggccaagataatctgggtattctatgtatccaagatccccgagtttattgacacgttcatcatggtcatcaaaaaaaacaaccgccagatctccttcctgcacatctaccaccactgctccatctttggcgtctggtggttcgtgttccttcaagccccaaacggagatgcctacttctcagctgcccttaactcctacatccacgtgatcatgtacgggtactacttcctatcctcaatcggagtgaagcaggtcagcttcgttaagcggtacatcaccatgtcccagatgacccagtttatgctcaacttctttcaggcctcttataatattgttgattgcttatacctccgccccgagcagtacgccaggggtgagctctaccctctcaacttgagcgtcatcctttggttctatatgatctcgatgctcggacttttctacaacttctttgttcaggatcgtcgtcgcgtccttgctgagaagaaggctgccacctatggcaaaaagcgcaactaagcggccgc

SEQ ID NO: 6 - protein, 326 aa, Coniodiobolus obscurus
MASAVYEKAASGMVPAAYYEKPADLIIEYVGRGLNYAAPLTQAVEGALIKAMPEAYSTVTNYLATTRSPLSEGFPLMNPVQVFLVMVSYLTIVFVGKAIMSNFTRIEAKTFSLFHNFAMVSISAYMCYGVVVQALADKYTLFTNPGDNTATGYPMAKIIWVFYVSKIPEFIDTFIMVIKKNNRQSFFHVYHHCSIFGVWWFVFLQAPNGDAYFSAALNSYIHVIMYGYYFLSSIGVKQVSFVKRYITMSQMTQFMLNFFQASYNIVDCLYLRPEQYARGELYPLNLSVILWFYMISMLGLFYNFFVQDRRRVLAEKKAATYGKKRN

SEQ ID NO: 7 - DNA, 1254 bp, Coniodiobolus thromboids
aaattttttccgggttttgttttactttcttattcaatgtaataaaagtatcaacaaaaaattgttaatatacctctatactttaacgtcaaggagaaaaaaccccggatcggactactagcagctgtaatacgactcactatagggaatattaagctcgcccttggaattcgaaatgagtttattaaataccttggatactattgcttcaagtaataacgttgtatcggcatataatgataccccagtagactatttaattaaagtagtagatttagctttaactactaacaaagcagtcttcaatgttgtagaagctaaaattaacgtatggatgccaacattgatgataaatttaagagaacagacttctagtttaatctcaccaataagtaaatatttgccattgttagatcctatccaagtgttttctattttgtttttatatatctttgttgtgtttttttggcttaaagtagcttctagcttcctcccacgtttcgaagtaagattattttcccttttccataatttctgtatggtcgttttatctgcctacatgtgctcttctatcctattacaagcttatgcagataagtatactctattcactaaccccgtcgatcactctccaaatggtattccaatggctaaaataatatggttattttatatttccaaaatcccagagtttgttgacactatgattatgttgattaaacaaaactaccgtcaaatttcctttttacatgtataccatcatagttcgatctttgctatctggtgggttgttaccttgatggcaccaaatggcgatgcttatttctcagctgcattgaattcatttattcatgttgttatgtatggatattatttactctctgcacttggattcaaatctgtctcctttgttaagaaatatattaccatggggcaaatgactcaatttgcactcaactttattcaagctagttataatattgtagacagaaattacttacgtccacaagtccatgagcaaggattagcttatccttatgctctttccgttttactttggttctatatgatctctatgttggtgttattcgctaatttttatattcaagatcgtatccgtcaatcaaagttaaagtctcaacaaaagggaaagaaaatgaattaggaattccaagggcgagcttcgaggtcacccattcgaaggtaagcctatccctaaccctctcctcggtctcgatctacgcgtaccgttca

SEQ ID NO: 8 - protein, 329 aa, Coniodiobolus thromboids
MSLLNTLDTIASSNNVVSAYNDTPVDYLIKVVDLALTTNKAVFNVVEAKINVWMPTLMINLREQTSSLISPISKYLPLLDPIQVFSILFLYIFVVFFWLKVASSFLPRFEVRLFSLFHNFCMVVLSAYMCSSILLQAYADKYTLFTNPVDHSPNGIPMAKIIWLFYISKIPEFVDTMIMLIKQNYRQISFLHVYHHSSIFAIWWVVTLMAPNGDAYFSAALNSFIHVVMYGYYLLSALGFKSVSFVKKYITMGQMTQFALNFIQASYNIVDRNYLRPQVHEQGLAYPYALSVLLWFYMISMLVLFANFYIQDRIRQSKLKSQQKGKKMN

SEQ ID NO: 9 - DNA, 1419 bp, Puccinia graminis
atggctacaaccgctctacctacctccgatcgagtcggtttggtatctagaccccctggaaaagttatccatctcacctcctccccatcctcctcgaccaggtcctcctcacccgactccttaaccaacgagaaggatggtatcgcaaccttcgaattaccagagtttactatcaaagagttattaggctcgatccctgcccactgcttcgaaagatccctcttcaaaagctcactctatgtcctcagggacttaagcttcacactcgctctgatctacttgggtcgtcagatcgatcccaacttcaacagcgtcgacggccaactcgtcaacggccgaccaggtgctatcttgaatttccttgcttgggccttttatggctactggatgggtttggtttggaccggtatctgggtcttagcgcatgagtgtggtcaccagtccttctccccctccaaatcgatcaacaacgccgtcggctgggtcttgcactctgccctcttggtacccttccactcctggcgaatcactcatgcccaacaccatgccgcaacctgccacatgaagcgagatcaggcttttgtgccttacactcgctcccagcttggccttcccccactagcagaaggttgcagcaagaaagaggtcgaagggtctcatccccctagcttttacgagagagtggacgatttactcgaggatgctcccctctgggctttgtacaagttgatcattcaccaatccattggattcgctagttacttgttgatcaatgcgtcaggccaaaagcactatccccgttggtggacaaatcacttcaacccttatgctatcatgtttgatgagcgccatcgatcccaagttgtttggtcggaccttggtattgctatcactattagctgcttgacttatcttggcaaacagaccgatttcttgacggtcttcaagtattacatcgtgccctacctcgtagttcatcactggattgtgttgatcactttcttgcaacacaccgaccctttactacctcattatcgtgagggagctttcaactttcaacggggtgcaatgtccaccatggacagaaacattcatggatttttcacccatggtttggcggaaacccatgtcgcccatcacttgtgctccaaaatcccccactacaacgcctgggaagcaaccgacgcattgaaagccaagttaggagaccattactcttccactgatgagaatttctggtacagcctttggaaatgcttccgacaatgtcgctttgttgaagatgaaggtgatgtggtattctacaaggatgctagaggcaaagctgccaggcgatacgtaccgaggaacactggtagtagtagtgtcagcgactcgggggttgatgtagacgggtctatcaaagaaaattcactatag

SEQ ID NO: 10 - protein, 472 aa, Puccinia graminis
MATTALPTSDRVGLVSRPPGKVIHLTSSPSSSTRSSSPDSLTNEKDGIATFELPEFTIKELLGSIPAHCFERSLFKSSLYVLRDLSFTLALIYLGRQIDPNFNSVDGQLVNGRPGAILNFLAWAFYGYWMGLVWTGIWVLAHECGHQSFSPSKSINNAVGWVLHSALLVPFHSWRITHAQHHAATCHMKRDQAFVPYTRSQLGLPPLAEGCSKKEVEGSHPPSFYERVDDLLEDAPLWALYKLIIHQSIGFASYLLINASGQKHYPRWWTNHFNPYAIMFDERHRSQVVWSDLGIAITISCLTYLGKQTDFLTVFKYYIVPYLVVHHWIVLITFLQHTDPLLPHYREGAFNFQRGAMSTMDRNIHGFFTHGLAETHVAHHLCSKIPHYNAWEATDALKAKLGDHYSSTDENFWYSLWKCFRQCRFVEDEGDVVFYKDARGKAARRYVPRNTGSSSVSDSGVDVDGSIKENSL

SEQ ID NO: 11 - protein, 6 aa, Artificial Sequence
  heme-binding site
XXHPGG

SEQ ID NO: 12 - protein, 7 aa, Artificial Sequence
  third histidine box
WFHGGLQ

SEQ ID NO: 13 - protein, 7 aa, Artificial Sequence
  histidine rich motif
FLHVYHH

SEQ ID NO: 14 - protein, 8 aa, Artificial Sequence
  tyrosine rich motif
MYTYYFLS

SEQ ID NO: 15 - DNA, 28 bp, Artificial Sequence
  primer LT53
atcttggtgcgcatatagcatgtggttc

SEQ ID NO: 16 - DNA, 29 bp, Artificial Sequence
  primer LT59
ggatccttaatcctgtttaggaggttcag

SEQ ID NO: 17 - DNA, 29 bp, Artificial Sequence
  primer LT60
gcggccgcattatggcctcagcagtttac

SEQ ID NO: 18 - DNA, 27 bp, Artificial Sequence
  primer LT57
aaccaccagacgccaaagatggagcag

SEQ ID NO: 19 - DNA, 21 bp, Artificial Sequence
  degenerate oligonucleotide forward primer LT14
ytgnarnccnccrrgraacca

SEQ ID NO: 20 - DNA, 20 bp, Artificial Sequence
  degenerate oligonucleotide reverse primer LT16
athgmnrancayccnggngg

SEQ ID NO: 21 - protein, 7 aa, Artificial Sequence
  conserved amino acid region of delta-6 desaturase enzymes
WFHGGLQ

SEQ ID NO: 22 - protein, 7 aa, Artificial Sequence
  conserved amino acid region of delta-6 desaturase enzymes
IXXHPGG

SEQ ID NO: 23 - protein, 28 aa, Artificial Sequence
  primer LT50
AGAGTTCCATAGCGTTCTCGGACCAGGC

SEQ ID NO: 24 - protein, 29 aa, Artificial Sequence
  primer LT51
TGCCATCCACATTGTTGAAAGAAGAGTCC

SEQ ID NO: 25 - protein, 26 aa, Artificial Sequence
  primer LT52
TGGGTTGGGGGTCACTTCTTTGGAGC

SEQ ID NO: 26 - protein, 28 aa, Artificial Sequence
  primer LT53
ATCTTGGTGCGCATATAGCATGTGGTTC

SEQ ID NO: 27 - DNA, 27 bp, Artificial Sequence
  primer LT58
ggatccatcatggcacctcttactaac

SEQ ID NO: 28 - DNA, 29 bp, Artificial Sequence
  primer LT59
ggatccttaatcctgtttaggaggttcag

SEQ ID NO: 29 - DNA, 20 bp, Artificial Sequence
  degenerate oligonucleotide primer LT5
ttyttncaygtntaycayca

SEQ ID NO: 30 - DNA, 20 bp, Artificial Sequence
  degenerate oligonucleotide primer LT6
arraartartanccrtacat

SEQ ID NO: 31 - protein, 7 aa, Artificial Sequence
  conserved amino acid region of delta-6 elongase enzymes
FLHVYHH

SEQ ID NO: 32 - protein, 8 aa, Artificial Sequence
  conserved amino acid region of delta-6 elongase enzymes
MYTYYFLS

SEQ ID NO: 33 - protein, 28 aa, Artificial Sequence
  primer LT56
ATCACGTGGATGTAGGAGTTAAGGGCAG

SEQ ID NO: 34 - protein, 27 aa, Artificial Sequence
  primer LT57
AACCACCAGACGCCAAAGATGGAGCAG

SEQ ID NO: 35 - protein, 26 aa, Artificial Sequence
  primer LT54
TCTTCCACGTCTACCACCACTGCTCC

SEQ ID NO: 36 - protein, 28 aa, Artificial Sequence
  primer LT55
TCAGCTGCCCTTAACTCCTACATCCACG

SEQ ID NO: 37 - DNA, 29 bp, Artificial Sequence
  primer LT60
gcggccgcattatggcctcagcagtttac

SEQ ID NO: 38 - DNA, 30 bp, Artificial Sequence
  primer LT61
gcggccgcttagttgcgctttttgccatag

SEQ ID NO: 39 - DNA, 30 bp, Artificial Sequence
  forward primer LT48
gaagatcttcgaaatggctaacaaatctcc

SEQ ID NO: 40 - DNA, 28 bp, Artificial Sequence
  reverse primer LT49
gaagatcttcctagccgtgtgtgtggac